(19) United States
(12) Patent Application Publication
(10) Pub. No.: US 2023/0286975 A1
(43) Pub. Date: Sep. 14, 2023

(54) IMPROVED METHOD FOR THE PRODUCTION OF LYSERGIC ACID DIETHYLAMIDE (LSD) AND NOVEL DERIVATIVES THEREOF

(71) Applicant: COMPASS PATHFINDER LIMITED, Altrincham, Cheshire (GB)

(72) Inventor: Matthias GRILL, Basel (CH)

(21) Appl. No.: 18/014,858

(22) PCT Filed: Jul. 7, 2021
(86) PCT No.: PCT/EP2021/068919

§ 371 Date: Jan. 6, 2023

(30) Foreign Application Priority Data
Jul. 7, 2020 (DE) 102020117924.3

(51) Int. Cl. C07D 457/08; C07D 519/00

(52) U.S. Cl. CPC C07D 457/08; C07D 519/00

(57) ABSTRACT

The present invention provides an improved method for the production of lysergic acid diethylamide (LSD) for GMP purposes. Furthermore, the present invention provides novel LSD derivatives of formula I as well as their synthesis and purification. Due to the affinity of the presented substances for the 5-HT_{2A} receptor, the invention may find application in numerous forms of therapy, such as against depression or drug addiction.

The present invention provides an improved method for the production of lysergic acid diethylamide (LSD) for GMP purposes. Furthermore, the present invention provides novel LSD derivatives of formula I as well as their synthesis and purification. Due to the affinity of the presented substances for the 5-HT_{2A} receptor, the invention can find application in numerous forms of therapy, such as, e.g., against depression or drug addiction.

### BACKGROUND

Lysergic acid diethylamide, often abbreviated as LSD, is a chemically produced derivative of lysergic acid, which naturally occurs in ergot alkaloids. LSD is one of the strongest known hallucinogens. Even in very small doses it evokes long lasting pseudohallucinogenic effects. Pharmacologically, LSD belongs to the group of serotonin-related psychedelic substances.

Research on hallucinogens has been experiencing a revival since about 1990.

In December 2007, for example, the Swiss psychiatrist Peter Gasserwas approved to conduct a double blind placebo-controlled phase II dose-effect pilot study of the psychotherapeutical treatment with LSD of patients suffering from end stage cancer. The results were promising but the experimental group of 12 persons was too small to be statistically representative.

More current publications are discussing LSD as a potential drug against cluster headache and migraine.

Recent studies are investigating the treatment of various diseases, such as anxiety disorders, ADHD, or depression by “microdosing”, i.e. the administration of small doses, which do not elicit hallucinations in the amounts used and the dosage interval of which is in the range of days or even weeks.

However, the currently available methods for the production of lysergic acid diethylamide (LSD) are using chlorinated solvents and/or carcinogenic catalysts.

At the same time, the yields and levels of purity of the lysergic acid diethylamides produced by these methods are less than optimal. Impurities, in particular with iso-LSD, do, however, represent a problem when using LSD as an active substance, for example against such most diverse pathologies as Parkinson's disease, dementia, and migraine.

One of the four stereoisomers [(+)-LSD or (5R,8R)-LSD] acts as a partial agonist of great affinity (binding strength) at the serotonin 5-HT_{2A} receptor. This receptor is associated with the mechanism of action of many atypical neuroleptic drugs. This, however, is no selective binding; a number of other receptor subtypes of the 5-HT-receptors, the dopamine receptors, and the adrenoceptors bind to LSD as well.

Due to this unspecific binding, numerous side effects are elicited, which prohibit a broader use of LSD-based pharmaceuticals.

A limited range of LSD derivatives have been described in the literature (see, e.g., Wagmann L et al., Analytical and Bioanalytical Chemistry, 2019, 411(19):4751-4763; and Halberstadt A L et al., Neuropharnacology, 2020, 172:107856) but none of these derivatives has resulted in the development of a successful pharmaceutical product.

Novel LSD derivatives, in particular those which have been shown to be selective modulators, i.e. which target a certain profile of neuro-receptors, are of great pharmaceutical interest even today.

The present invention addresses the above-discussed shortcomings in the state of the art and solves the problem of providing an improved method of preparing LSD, which allows the production of LSD in advantageously high purity and yield as well as in compliance with GMP requirements. The LSD produced by the method according to the invention is particularly well suited for use in therapy. Moreover, the present invention also solves the problem of providing novel and/or improved LSD derivatives which exhibit highly advantageous properties, including in particular with respect to their pharmacological activity and their receptor subtype selectivity, and further provides a method of preparing such LSD derivatives with high purity and yield, including high isomeric purity.

### DESCRIPTION OF THE INVENTION

The present invention relates to a novel method for the conversion of lysergic acid (1) into a mixture of d-lysergic acid diethylamide (2a) and d-iso-lysergic acid diethylamide (2b):

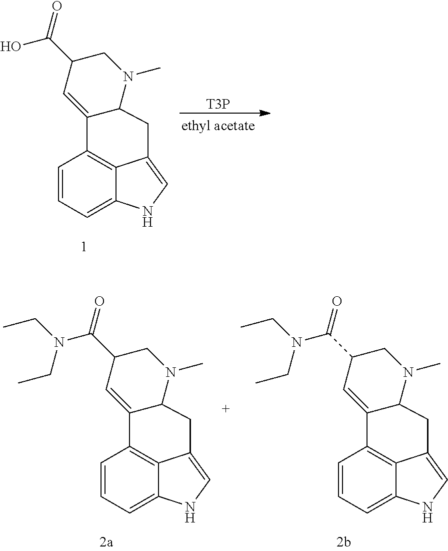

Based on the use of a specific combination of the coupling agent propane-phosphonic acid anhydride (T3P) and ethyl acetate as a solvent, it was surprisingly found that a quantitative conversion with significantly less by-products is feasible and that the yield and/or purity are significantly increased.

In particular, and without being bound by theory, it has surprisingly been found that the use of ethyl acetate as a solvent is highly advantageous, as it results in the intermediate formation of lysergic acid ethyl ester which is completely soluble in the reaction mixture (in contrast to lysergic acid which is hardly soluble) and thus allows a quantitative conversion with diethylamine into lysergic acid diethylamide.

Moreover, in comparison to all methods of production according to the state of the art, another advantageous feature of the present method of production is that it dispenses with chlorinated solvents and/or carcinogenic catalysts, which results in a greatly improved environmental compatibility of the selected reagents and solvents. Accordingly, the methods provided herein, including the above-described method for the conversion of lysergic acid into d-LSD and d-iso-LSD, as well as the method for the production of LSD or an LSD derivative (as described herein below), can be conducted without using any chlorinated solvents and/or without using any carcinogenic catalysts.

The method of production of the present invention is also usable in GMP production, as an improved yield of at least 85% and without impurities of d-iso-lysergic acid diethylamide can be achieved.

In a first aspect, the present invention relates to a novel method for the production of lysergic acid diethylamide (LSD) or a derivative thereof, comprising the steps of:
a. preparing a suspension of lysergic acid hydrate in ethyl acetate;
b. addition of an amine compound (e.g., diethylamine) under protective gas atmosphere;
c. addition of propane-phosphonic acid anhydride solution (T3P) in ethyl acetate;
d. stirring of the mixture under protective gas atmosphere for at least 4 hours;
e. stopping the reaction by dilution with ethyl acetate,
f. extraction with water,
g. drying of the organic phase over a desiccant at 20-60° C. and under vacuum,
h. obtaining a crude product containing lysergic acid diethylamide (LSD) or a derivative thereof.

It will be understood that the use of diethylamine in step b. of this method allows the production of lysergic acid diethylamide (LSD), whereas an LSD derivative (in which the diethylamide group contained in LSD is modified) can be obtained by using an amine compound other than diethylamine in step b.

Thus, in accordance with the above, the invention provides a method for the production of lysergic acid diethylamide (LSD), comprising the steps of:
a. preparing a suspension of lysergic acid hydrate in ethyl acetate;
b. addition of diethylamine under protective gas atmosphere;
c. addition of propane-phosphonic acid anhydride solution (T3P) in ethyl acetate;
d. stirring of the mixture under protective gas atmosphere for at least 4 hours;
e. stopping the reaction by dilution with ethyl acetate,
f. extraction with water,
g. drying of the organic phase over a desiccant at 20-60° C. and under vacuum,
h. obtaining a crude product containing lysergic acid diethylamide (LSD).

Moreover, the invention also provides a method for the production of an LSD derivative, particularly an LSD derivative of the following formula II

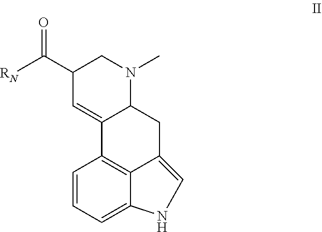

wherein the group R_{N} is as defined below, comprising the steps of:
a. preparing a suspension of lysergic acid hydrate in ethyl acetate;
b. addition of an amine compound, particularly an amine compound of the formula R_{N}—H wherein R_{N} has the same meaning as in formula II, under protective gas atmosphere;
c. addition of propane-phosphonic acid anhydride solution (T3P) in ethyl acetate;
d. stirring of the mixture under protective gas atmosphere for at least 4 hours;
e. stopping the reaction by dilution with ethyl acetate,
f. extraction with water,
g. drying of the organic phase over a desiccant at 20-60° C. and under vacuum,
h. obtaining a crude product containing the LSD derivative, particularly the LSD derivative of formula II.

In formula II, the group R_{N} may be any secondary or tertiary amino group (resulting in a secondary or tertiary amide when attached to the carbonyl (—CO—) group in formula II), or R_{N} may be any N-containing heterocyclyl group which comprises at least one nitrogen ring atom, which is attached to the remainder of the compound of formula II via said nitrogen ring atom, and which may be optionally substituted (e.g., with one or more groups R₄, as defined below).

The amine compound of the formula R_{N}—H can be chosen so as to obtain the corresponding LSD derivative having the same group R_{N} attached to the carbonyl (—CO—) group in formula II. Accordingly, the group R_{N} in the amine compound of the formula R_{N}—H has the same meaning as the group R_{N} in the LSD derivative of formula II. For example, if R_{N} is a group —NH—CH₂—CHF₂, then the amine compound of the formula R_{N}—H is a compound H₂N—CH₂—CHF₂.

Preferably, the group R_{N} (in the LSD derivative of formula II and in the amine compound of the formula R_{N}—H) is selected from —NH—(C₁₋₅ alkyl), —N(C₁₋₅ alkyl)(C₁₋₅ alkyl), —NH—(C₁₋₅ haloalkyl), —N(C₁₋₅ alkyl)(C₁₋₅ haloalkyl), —N(C₁₋₅ haloalkyl)(C₁₋₅ haloalkyl), —NH—(C₁₋₅ alkylene)-O—(C₁₋₅ alkyl), —N(C₁₋₅ alkyl)-(C₁₋₅ alkylene)-O—(C₁₋₅ alkyl), —N[—(C₁₋₅ alkylene)-O—(C₁₋₅ alkyl)]-(C₁₋₅ alkylene)-O—(C₁₋₅ alkyl), —N(C₁₋₅ haloalkyl)-(C₁₋₅ alkylene)-O—(C₁₋₅ alkyl), —N(C₃₋₇ cycloalkyl)(C₃₋₇ cycloalkyl), an N-containing polycyclic heterocyclyl, 1,3-oxazolidin-3-yl, 3-methylpyrrolidin-1-yl, and an N-containing monocyclic heterocyclyl which is substituted with one or more halogens, wherein any alkyl groups and/or any alkylene groups comprised in said —NH—(C₁₋₅ alkylene)-O—(C₁₋₅ alkyl), in said —N(C₁₋₅ alkyl)-(C₁₋₅ alkylene)-O—(C₁₋₅ alkyl) or in said —N[—(C₁₋₅ alkylene)-O—(C₁₋₅ alkyl)]-(C₁₋₅ alkylene)-O—(C₁₋₅ alkyl) are each optionally substituted with one or more halogens, wherein said N-containing polycyclic heterocyclyl or said N-containing monocyclic heterocyclyl comprises at least one nitrogen ring atom and is attached to the remainder of the compound via said nitrogen ring atom, wherein said N-containing polycyclic heterocyclyl is not indolin-1-yl, 1,2,3,4-tetrahydroquinolin-1-yl or 3-azabicyclo[3.2.2]nonan-3-yl, wherein said N-containing polycyclic heterocyclyl, said 1,3-oxazolidin-3-yl, said N-containing monocyclic heterocyclyl, and the cycloalkyl groups comprised in said —N(C₃₋₇ cycloalkyl)(C₃₋₇ cycloalkyl) are each optionally substituted with one or more groups R₄ (wherein R₄ is as defined herein below), and further wherein R_{N} is not —N(CH₂CH₃)—CH₂CH₃.

More preferably, the group R_{N} has the same meaning as the group R₁ in formula I, as described and defined herein below, including the general (broadest) meaning of R₁ or any specific (exemplary or preferred) meaning of R₁.

The following description relates to the above-described method for the production of lysergic acid diethylamide (LSD) or a derivative thereof, including specifically the above-described method for the production of LSD and specifically the above-described method for the production of an LSD derivative.

In one embodiment, in step a., between 5 mmol and 20 mmol lysergic acid hydrate are suspended in 50 ml up to 500 ml ethyl acetate. Other carboxylic acid esters are possible as well, but ethyl acetate is preferred as it is environmentally compatible, low-priced and nontoxic. It is also advantageous that the impurities to be potentially characterized in a GMP material can only be lysergic acid ethyl ester and acetic acid when ethyl acetate is used. In one embodiment, 10 mmol (2.86 g) lysergic acid hydrate are suspended in 200 ml ethyl acetate.

Steps a. to f. may be carried out at any suitable temperature, e.g., at a temperature of 4° C. to 80° C. Preferably, steps a. to f. are carried out at a temperature of 10 to 60° C., more preferably at 15° C. to 40° C., even more preferably at 20° C. to 30° C., still more preferably at about 25° C. Accordingly, in one embodiment, steps a. to f. are carried out at 20-30° C., preferably at 25° C. In one embodiment, steps a.-c. are carried out at 25° C. and step d. at 26° C.

In one embodiment, in step b., 20 mmol to 300 mmol of the amine compound (i.e., diethylamine for the production of LSD, or another amine compound for the production of an LSD derivative) are added. Preferably, between 50 mmol and 200 mmol diethylamine (or another amine compound) are added. In one embodiment, 100 mmol (10.4 ml) diethylamine are added. The suspension is aerated with protective gas.

The term “protective gas”, as used herein, refers to aeration with an inert gas, preferably argon. In other embodiments, also a different protective gas can be employed, e.g. elementary gases such as nitrogen, noble gases such as helium, neon, argon, krypton, xenon, and gaseous molecular compounds like sulfur hexafluoride.

In one embodiment, in step c., between 30 wt. % and 65 wt. % propane-phosphonic acid anhydride solution (T3P) in ethyl acetate (e.g., in 10 mmol to 300 mmol, preferably in 25 mmol to 35 mmol) are added, preferably dropwise through a septum. T3P dissolved in other solvents (e.g. DMF) can also be used (instead of T3P dissolved in ethyl acetate), but the ethyl acetate solution is preferred as it is nontoxic and environmentally compatible, and as undesired impurities can be avoided in synthesis. The drip rate is preferably adjusted such that the addition takes between 50 and 90 minutes. In one embodiment, in step c., 50 wt. % propane-phosphonic acid anhydride solution (T3P) in ethyl acetate (30 mmol, 19.08 g) are added dropwise through a septum during 70 minutes (i.e., over the course of 70 minutes).

In step d., the mixture is stirred under protective gas atmosphere for at least 4 hours, e.g., for 4 hours to 10 days, preferably for 4 to 48 hours, more preferably for 4 to 24 hours, even more preferably for 4 to 8 hours (e.g., for about 5 hours). The temperature at which the mixture is stirred may be, for example, 4° C. to 80° C., preferably 10 to 60° C., more preferably 15° C. to 40° C., even more preferably 20° C. to 30° C., still more preferably about 25° C. In one embodiment, in step d., the mixture is stirred between 4 and 24 hours, preferably between 4 and 8 hours at 20-28° C. under protective gas atmosphere. In one embodiment, in step d., the mixture is stirred for about 5 hours at 26° C.

In a further embodiment, in step e., the reaction is stopped by adding between 100 ml and 500 ml ethyl acetate. In one embodiment, in step e., the reaction is stopped by adding 200 ml ethyl acetate.

In a further embodiment, in step f., extraction of the mixture is performed with between 75 and 450 ml water. In one embodiment, extraction of the mixture is performed with 150 ml water. The pH value of the aqueous phase should be in an alkaline range. In one embodiment, it is at pH 7.5 to 13, preferably at pH 7.5 to 12, more preferably at pH 8 to 10, in a further embodiment at pH 9.

In a further embodiment, in step g., the mixture is dried. The drying of the organic phase over a desiccant may be conducted under vacuum (reduced pressure) at a temperature of 20° C. to 60° C., preferably at 35° C. to 60° C., more preferably at 40° C. to 60° C., even more preferably at 40° C. to 50° C., still more preferably at about 45° C. The pressure is preferably 20 to 700 mbar, more preferably 30 to 600 mbar (e.g., 30 to 60 mbar), even more preferably 100 to 500 mbar, still more preferably 200 to 300 mbar. It is particularly preferred that the drying in step g. is conducted at 40° C. to 60° C. (particularly at about 45° C.) and at a pressure of 200 to 300 mbar. Also particularly preferred is drying with a desiccant at a temperature of between 35° C. and 60° C. and under a vacuum (reduced pressure) of 30-60 mbar. Preferred desiccants are anhydrous calcium chloride, anhydrous sodium carbonate, anhydrous potassium carbonate, anhydrous sodium sulfate, anhydrous magnesium sulfate, or anhydrous calcium sulfate. In one embodiment, the desiccant is anhydrous MgSO₄, the temperature is 45° C. and the vacuum is 40 mbar.

The methods according to the invention, including the method for the production of LSD and the method for the production of an LSD derivative, are advantageous in that they do not require any chlorinated or halogenated solvents. It is thus preferred that the method for the production of LSD or the method for the production of an LSD derivative is conducted without using any chlorinated solvents, more preferably without using any halogenated solvents. Moreover, it is preferred that any of these methods is conducted without using any auxiliary base (i.e., without using any base other than diethylamine or other than the amine compound).

In the method for the production of LSD, the crude product obtained in step h. (i.e., after carrying out steps a. to g.) is typically a yellow-brown oil. The crude product contains LSD and iso-LSD.

In one embodiment, an HPLC measurement at 312 nm after a reaction of 2.5 h shows a yield of about 0.85% lysergic acid diethylamide, wherein about 70% of this is LSD and about 30% is iso-LSD.

In further embodiments, after 4 hours the HPLC measurement at 312 nm shows in the crude product more than 90%, preferably more than 95%, more preferably more than 99%, in a particularly preferred embodiment 99.9% product and traces of ethyl ester, in one embodiment for example 0.1% ethyl ester.

Conventional methods only achieve yields of about 50-70%.

In a further aspect of the present invention, the crude product is subsequently subjected to a method for isomer optimization, comprising the steps of:
a. dissolving the crude product in ethanol,
b. addition of sodium methoxide,
c. stirring for at least 2 hours,
d. dilution with water,
e. distillation of the solvent,
f. redilution of the residue with water,
g. extraction with ethyl acetate,
h. drying of the organic phase over a desiccant and under vacuum,
i. obtaining the isomer-optimized intermediate product.

In one embodiment, in step a., the crude product is dissolved in 30-100 ml ethanol (abs.). In principle, other alcohols can also be used, for example isopropanol, but, here too, those alcohols are preferable, which are nontoxic and environmentally compatible in order to ensure their suitability for synthesis for GMP purposes. In a further embodiment, in step a., the crude product is dissolved in 60 ml ethanol (abs.).

The abbreviation “GMP”, as used herein, refers to Good Manufacturing Practice. This term describes regulations on quality assurance of the production processes and environments in the manufacture of medicinal products and active substances, as well as of cosmetics, food, and feed products. Quality assurance plays a central role in the manufacturing of pharmaceutical products since any lessening in quality can directly affect the health of the patients or consumers. A quality management system in accordance with GMP guarantees product quality and compliance with requirements set by the health authorities for the sale of products.

Thus, an important aspect of the present invention is that the novel method of production is GMP-compliant.

In one embodiment, in step b., 350-600 mg sodium methoxide (CH₃ONa) are added. As catalyst, other bases, such as sodium hydroxide (NaOH) or potassium hydroxide (KOH), may also be used. In a preferred embodiment, the whole process is anhydrous. In a further embodiment, in step b., 520 mg sodium methoxide (CH₃ONa) are added. Not too much base should be added since it affects the LSD.

In one embodiment, in step c., the mixture is stirred for at least 2 hours, preferably at least 2 hours up to 3 hours, preferably at least 2 hours up to 4 hours, preferably at least 2 hours up to 6 hours. The mixture may be stirred at a temperature of 4° C. to 80° C., preferably at 20 to 70° C., more preferably at 40-60° C., even more preferably at 50° C. In a further embodiment, in step c., the mixture is stirred for about 3 hours at 50° C. It is particularly preferred that the mixture is stirred for about 4 hours at 50° C. The stirring should preferably not last longer than 12 hours since long stirring periods, especially when using a base, affect the LSD. Following 2-4 hours of stirring an equilibrium of >80% LSD is reached. The ratio of LSD to iso-LSD is not improving with markedly longer stirring periods of 12 hours or even 24 hours, but first decompositions of the LSD become visible in HPLC.

In one embodiment, in step d., the mixture is diluted with 50-200 ml water. In a further embodiment, the mixture is diluted with 100 ml water.

In one embodiment, in step e., the solvent is distilled. In a further embodiment, the solvent is distilled on a rotary evaporator (e.g., at 50° C., 90 mbar).

In one embodiment, in step f., the aqueous residue is rediluted with 50-200 ml water. In a further embodiment, in step f., the aqueous residue is rediluted with 100 ml water. As an initial dilution with water is already conducted in step d., the subsequent dilution with water in step f. is referred to as “redilution” (or further dilution).

In one embodiment, in step g., the aqueous residue is extracted with 100-200 ml ethyl acetate at least once, preferably at least twice, more preferably at least three times. In a further embodiment, in step g., the aqueous residue is extracted four times with 150 ml ethyl acetate.

In a further embodiment, in step h., the mixture is dried and concentrated.

In particular, the drying of the organic phase over a desiccant and under vacuum (in step h.) may be conducted at a temperature of 20° C. to 70° C., preferably at 35° C. to 60° C., more preferably at 40° C. to 50° C., even more preferably at about 45° C. The drying may be conducted at a vacuum (reduced pressure) of 20 mbar to 700 mbar, preferably 25 mbar to 300 mbar (e.g., 200 to 300 mbar), more preferably 30 mbar to 60 mbar, even more preferably about 40 mbar. Particularly preferred is drying with a desiccant and concentrating at a temperature of between 35° C. and 60° C. and a vacuum of 30-60 mbar. In one embodiment, the desiccant is anhydrous MgSO₄, the temperature is 45° C. and the vacuum is 40 mbar.

In step i., typically a brown oil is obtained.

In one embodiment, the intermediate product is set to a ratio of d-lysergic acid diethylamide (2a):d-iso-lysergic acid diethylamide (2b) of 84%:16% by the method described herein.

Iso-LSD and other impurities are preferably removed in a subsequent column chromatographic purification process.

In one embodiment, the isomer-optimized intermediate product is column treated over 300 g silica using the eluent mixture toluene/ethanol in a ratio of 95:5. Other column materials known to the person skilled in the art can be employed as well.

Alternatively, basic aluminum oxide (AlOx) (activity II) with benzene/dichloromethane as an eluent or basic AlOx (II-III) with chloroform/toluene could be employed as well, however, the separation is worse in this case.

The isomer-optimized intermediate product is dissolved and applied in toluene/ethanol, in one embodiment in a ratio of 70:30, in a volume of 5-20 ml, preferably in a volume of 15 ml. This purification process is particularly advantageous, as it does not require any chlorinated or halogenated solvents. Thus, it is preferred that the isomer-optimized intermediate product is subjected to a column chromatographic purification process using the eluent mixture toluene/ethanol, without using any chlorinated solvents, more preferably without using any halogenated solvents. Alternatively, dichloromethane/methanol could be employed in a ratio of 8:2 as well, but this could compromise the GMP compliance.

The column is started with the eluent mixture toluene/ethanol at 95:5. Following one liter of this eluent it is changed to toluene/ethanol at 90:10. One of the impurities runs first as a green band (365 nm). The purified product runs directly behind it as a bright violet region (365 nm).

The product fractions are concentrated, for example by means of a rotary evaporator.

In one embodiment, a theoretical yield of more than 80%, preferably more than 85%, particularly preferably of 90% and more, is achieved after having performed the three methods. In one embodiment, the theoretical yield is 89%.

The product may be reexamined by using high performance liquid chromatography, HPLC, at 312 nm. In one embodiment, a purity is achieved of more than 90%, preferably more than 95%, particularly preferably of 99%, and particularly preferably of 99.9%. In one embodiment, no iso-LSD at all is contained in the product anymore.

The method of production described herein is also extremely well-suited for the synthesis of LSD derivatives (including the LSD derivatives of formula II as well as the novel LSD derivatives of formula I described herein below) of high purity and/or at high yields. Thus, in a further aspect, the present invention also provides the following novel LSD derivatives, which can be synthesized using the above-described method of the invention or by further modifying the LSD or the LSD derivatives produced by the method of the invention.

The present invention thus provides compounds having the general formula I and pharmaceutically acceptable salts thereof, which can be produced in high yield and purity using the method according to the invention:

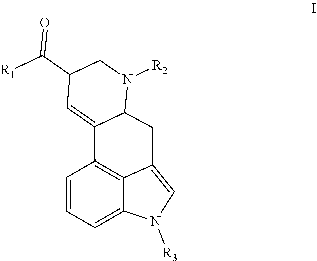

In formula I, the group R₁ is selected from —NH—(C₁₋₅ haloalkyl), —N(C₁₋₅ alkyl)(C₁₋₅ haloalkyl), —N(C₁₋₅ haloalkyl)(C₁₋₅ haloalkyl), —NH—CH₂—O—(C₁₋₅ alkyl), —NH—(CH₂)₃₋₅—O—(C₁₋₅ alkyl), —N(C₁₋₅ alkyl)[—(C₁₋₅ alkylene)-O—(C₁₋₅ alkyl)], —N[—(C₁₋₅ alkylene)-O—(C₁₋₅ alkyl)]-(C₁₋₅ alkylene)-O—(C₁₋₅ alkyl), —N(C₁₋₅ haloalkyl)[—(C₁₋₅ alkylene)-O—(C₁₋₅ alkyl)], —N(C₃₋₇ cycloalkyl)(C₃₋₇ cycloalkyl), an N-containing polycyclic heterocyclyl, 1,3-oxazolidin-3-yl, 3-methylpyrrolidin-1-yl, and an N-containing monocyclic heterocyclyl which is substituted with one or more halogens,

wherein said —NH—(C₁₋₅ haloalkyl) is not —NH—CH₂CH₂—Cl or —NH—CH(—CH₂CH₃)—CH₂—Cl, wherein any alkyl groups and/or any alkylene groups comprised in said —NH—CH₂—O—(C₁₋₅ alkyl), in said —NH—(CH₂)₃₋₅—O—(C₁₋₅ alkyl), in said —N(C₁₋₅ alkyl)[—(C₁₋₅ alkylene)-O—(C₁₋₅ alkyl)] or in said —N[—(C₁₋₅ alkylene)-O—(C₁₋₅ alkyl)]-(C₁₋₅ alkylene)-O—(C₁₋₅ alkyl) are each optionally substituted with one or more (e.g., one, two or three) halogens, wherein said N-containing polycyclic heterocyclyl or said N-containing monocyclic heterocyclyl comprises at least one nitrogen ring atom and is attached to the remainder of the compound of formula I via said nitrogen ring atom, wherein said N-containing polycyclic heterocyclyl is not indolin-1-yl, 1,2,3,4-tetrahydroquinolin-1-yl or 3-azabicyclo[3.2.2]nonan-3-yl, and further wherein said N-containing polycyclic heterocyclyl, said 1,3-oxazolidin-3-yl, said N-containing monocyclic heterocyclyl, and the cycloalkyl groups comprised in said —N(C₃₋₇ cycloalkyl)(C₃₋₇ cycloalkyl) are each optionally substituted with one or more (e.g., one, two or three) groups R₄; and
R₂ is selected from C₁₋₅ alkyl, C₂₋₅ alkenyl, C₂₋₅ alkynyl, and C₁₋₅ haloalkyl.

Alternatively, R₁ is —NH—(C₁₋₅ alkyl) or —N(C₁₋₅ alkyl)(C₁₋₅ alkyl), and R₂ is C₁₋₅ haloalkyl.

As explained above, R₁ may be an N-containing polycyclic heterocyclyl which comprises at least one nitrogen ring atom and is attached to the remainder of the compound of formula I (i.e., to the carbonyl group —C(═O)— shown in formula I) via said nitrogen ring atom (and which is optionally substituted with one or more R₄, as defined above); said N-containing polycyclic heterocyclyl is not indolin-1-yl, 1,2,3,4-tetrahydroquinolin-1-yl or 3-azabicyclo[3.2.2]nonan-3-yl. This N-containing polycyclic heterocyclyl may be, for example, an N-containing polycyclic heterocycloalkyl, an N-containing polycyclic heterocycloalkenyl, or an N-containing polycyclic heteroaryl. Preferably, the N-containing polycyclic heterocyclyl is an N-containing polycyclic heterocycloalkyl. It is furthermore preferred that any of the aforementioned polycyclic groups is a bicyclic group. Thus, more preferably, the N-containing polycyclic heterocyclyl is a bicyclic N-containing heterocycloalkyl, e.g., a fused bicyclic, a bridged bicyclic, or a spiro-bicyclic N-containing heterocycloalkyl; each ring comprised in said bicyclic N-containing heterocycloalkyl may independently have, e.g., 4, 5, 6 or 7 ring atoms. The N-containing polycyclic heterocyclyl (or the N-containing polycyclic heterocycloalkyl, including any of the aforementioned bicyclic N-containing heterocycloalkyls) comprises at least one nitrogen ring atom (which forms the attachment point of the group R₁) and optionally comprises one or more (e.g., one, two, three or four) further ring heteroatoms selected independently from nitrogen, oxygen and sulfur, while the remaining ring atoms are carbon atoms. It will be understood that each one of the aforementioned N-containing heterocycloalkyl groups is optionally substituted with one or more (e.g., one, two or three) groups R₄. Particularly preferred examples of an N-containing polycyclic heterocyclyl (as group R₁) include 2-oxa-6-azaspiro[3.3]-heptan-6-yl or 7-azabicyclo[2.2.1]hept-7-yl, wherein each of the aforementioned groups is optionally substituted with one or more groups R₄.

Moreover, as also explained above, R₁ may be an N-containing monocyclic heterocyclyl which is substituted with one or more halogens, wherein said N-containing monocyclic heterocyclyl comprises at least one nitrogen ring atom and is attached to the remainder of the compound of formula I (i.e., to the carbonyl group —C(═O)— shown in formula I) via said nitrogen ring atom (and which is optionally substituted with one or more R₄, as defined above). This N-containing monocyclic heterocyclyl (which is substituted with one or more halogens) may be, for example, an N-containing monocyclic heterocycloalkyl, an N-containing monocyclic heterocycloalkenyl, or an N-containing monocyclic heteroaryl. Preferably, the N-containing monocyclic heterocyclyl is an N-containing monocyclic heterocycloalkyl. More preferably, the N-containing monocyclic heterocyclyl is an N-containing monocyclic heterocycloalkyl having 4, 5, 6 or 7 ring atoms. The N-containing monocyclic heterocyclyl (or the N-containing monocyclic heterocycloalkyl) comprises at least one nitrogen ring atom (which forms the attachment point of the group R₁) and optionally comprises one or more (e.g., one or two) further ring heteroatoms selected independently from nitrogen, oxygen and sulfur, while the remaining ring atoms are carbon atoms. It will be understood that each one of the aforementioned N-containing monocyclic heterocycloalkyl groups is substituted with one or more (e.g., one, two or three) halogens and is optionally further substituted with one or more (e.g., one, two or three) groups R₄. A particularly preferred example of an N-containing monocyclic heterocyclyl substituted with one or more halogens is 3-fluoro-azetidin-1-yl.

Preferably, R₁ is selected from —NH—(C₁₋₅ haloalkyl), —N(C₁₋₅ alkyl)(C₁₋₅ haloalkyl), —N(C₁₋₅ haloalkyl)(C₁₋₅ haloalkyl), —N(C₁₋₅ haloalkyl)[—(C₁₋₅ alkylene)-O—(C₁₋₅ alkyl)], —N(—CH₃)—CH₂CH₂—O—CH₃, —N(cyclopropyl)(cyclopropyl), an N-containing polycyclic heterocycloalkyl, 1,3-oxazolidin-3-yl, 3-methylpyrrolidin-1-yl, and an N-containing monocyclic heterocycloalkyl which is substituted with one or more halogens,

wherein said —NH—(C₁₋₅ haloalkyl) is not —NH—CH₂CH₂—Cl or —NH—CH(—CH₂CH₃)—CH₂—Cl, wherein said N-containing polycyclic heterocycloalkyl or said N-containing monocyclic heterocycloalkyl comprises at least one nitrogen ring atom and is attached to the remainder of the compound of formula I via said nitrogen ring atom, wherein said N-containing polycyclic heterocycloalkyl is not 3-azabicyclo[3.2.2]nonan-3-yl, and further wherein said N-containing polycyclic heterocycloalkyl, said 1,3-oxazolidin-3-yl and said N-containing monocyclic heterocycloalkyl are each optionally substituted with one or more (e.g., one, two or three) groups R₄, and
R₂ is selected from C₁₋₅ alkyl (e.g., methyl), C₂₋₅ alkenyl (e.g., allyl, i.e., —CH₂—CH═CH₂), C₂₋₅ alkynyl (e.g., propargyl, i.e., 2-propynyl), and C₁₋₅ haloalkyl (e.g., —CH₂CH₂F);
or alternatively, R₁ is —NH—(C₁₋₅ alkyl) or —N(C₁₋₅ alkyl)(C₁₋₅ alkyl), and R₂ is C₁₋₅ haloalkyl (e.g., —CH₂CH₂F).

More preferably, R₁ is selected from —NH—(C₁₋₅ haloalkyl) (e.g., —NH—CH₂CF₃ or —NH—CH₂CF₂H), —N(C₁₋₅ alkyl)(C₁₋₅ haloalkyl) (e.g., —N(—CH₃)—CH₂CF₃ or —N(—CH₂CH₃)—CH₂CH₂F), —N(C₁₋₅ haloalkyl)(C₁₋₅ haloalkyl), —N(C₁₋₅ haloalkyl)[—(C₁₋₅ alkylene)-O—(C₁₋₅ alkyl)] (e.g., —N(—CH₂CF₂H)[—CH₂CH₂—O—CH₃]), —N(—CH₃)—CH₂CH₂—O—CH₃, —N(cyclopropyl)(cyclopropyl), an N-containing polycyclic heterocycloalkyl, 1,3-oxazolidin-3-yl, 3-methylpyrrolidin-1-yl, and an N-containing monocyclic heterocycloalkyl which is substituted with one or more halogens,

wherein said —NH—(C₁₋₅ haloalkyl) is not —NH—CH₂CH₂—Cl or —NH—CH(—CH₂CH₃)—CH₂—Cl, wherein said N-containing polycyclic heterocycloalkyl or said N-containing monocyclic heterocycloalkyl comprises at least one nitrogen ring atom and is attached to the remainder of the compound of formula I via said nitrogen ring atom, and wherein said N-containing polycyclic heterocycloalkyl is not 3-azabicyclo[3.2.2]nonan-3-yl, and
R₂ is C₁₋₅ alkyl or C₁₋₅ haloalkyl (it is particularly preferred that R₂ is methyl or —CH₂CH₂F);
or alternatively, R₁ is —NH—(C₁₋₅ alkyl) or —N(C₁₋₅ alkyl)(C₁₋₅ alkyl) (e.g., —N(—CH₂CH₃)—CH₂CH₃), and R₂ is C₁₋₅ haloalkyl (it is particularly preferred that R₂ is —CH₂CH₂F).

In accordance with the above, it is particularly preferred that R₂ is methyl or —CH₂CH₂F; or, if R₁ is —NH—(C₁₋₅ alkyl) or —N(C₁₋₅ alkyl)(C₁₋₅ alkyl), it is particularly preferred that R₂ is —CH₂CH₂F.

In embodiments of formula I, R₁ is an N-containing polycyclic heterocycloalkyl, wherein said N-containing polycyclic heterocycloalkyl comprises at least one nitrogen ring atom and is attached to the remainder of the compound of formula I via said nitrogen ring atom, wherein said N-containing polycyclic heterocycloalkyl is not 3-azabicyclo[3.2.2]nonan-3-yl, and further wherein said N-containing polycyclic heterocycloalkyl is optionally substituted with one or more (e.g., one, two or three) groups R₄; and

R₂ is selected from C₁₋₅ alkyl, C₂₋₅ alkenyl, and C₂₋₅ alkynyl, wherein the aforementioned groups are each optionally substituted with one or more (e.g., one, two or three) halogens.

In embodiments of formula I, R₁ is an —NH—(C₁₋₅ haloalkyl), wherein said —NH—(C₁₋₅ haloalkyl) is not —NH—CH₂CH₂—Cl or —NH—CH(—CH₂CH₃)—CH₂—Cl, and further wherein said —NH—(C₁₋₅ haloalkyl) is optionally substituted with one or more (e.g., one, two or three) groups R₄; and

R₂ is selected from C₁₋₅ alkyl, C₂₋₅ alkenyl, and C₂₋₅ alkynyl, wherein any groups in said are each optionally substituted with one or more (e.g., one, two or three) halogens.

R₃ is selected from hydrogen, C₁₋₅ alkyl, —CO—(C₁₋₅ alkyl), —CO—(C₃₋₆ cycloalkyl), and an amino acid, wherein said amino acid is attached via a —CO— group formed from a carboxylic acid group of the amino acid, and further wherein said C₁₋₅ alkyl, the alkyl group comprised in said —CO—(C₁₋₅ alkyl), the cycloalkyl group comprised in said —CO—(C₃₋₆ cycloalkyl) and any alkyl group comprised in said amino acid are each optionally substituted with one or more (e.g., one, two or three) halogens.

Preferably, R₃ is hydrogen, C₁₋₅ alkyl, —CO—(C₁₋₅ alkyl), or —CO—(C₃₋₆ cycloalkyl). More preferably, R₃ is hydrogen, —CO—(C₁₋₅ alkyl), or —CO—(C₃₋₆ cycloalkyl). Corresponding preferred examples of R₃ include hydrogen, —CO—CH₃, —CO—CH₂CH₃, —CO—CH₂CH₂CH₃, or —CO-cyclopropyl. Even more preferably, R₃ is hydrogen.

Each R₄ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—CO—O—(C₁₋₅ alkyl), —(C₀₋₃ alkylene)-N(C₁₋₅ alkyl)-CO—O—(C₁₋₅ alkyl), —(C₀₋₃ alkylene)-O—CO—NH—(C₁₋₅ alkyl), —(C₀₋₃ alkylene)-O—CO—N(C₁₋₅ alkyl)-(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO₂—(C₁₋₅ alkyl), and —(C₀₋₃ alkylene)-SO—(C₁₋₅ alkyl).

Preferably, each R₄ is independently selected from C₁₋₅ alkyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —NO₂, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—CO—O—(C₁₋₅ alkyl), —N(C₁₋₅ alkyl)-CO—O—(C₁₋₅ alkyl), —O—CO—NH—(C₁₋₅ alkyl), —O—CO—N(C₁₋₅ alkyl)-(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl).

Moreover, it is particularly preferred that the groups in the compound of formula I are defined as follows:

| R₁ | R₂ | R₃ |
|---|---|---|
| 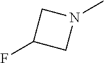 | CH₃ | H, or COCH₃, or COCH₂CH₃ |
| 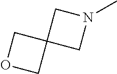 | CH₃ | H, or COCH₃, or COCH₂CH₃ |
| 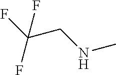 | CH₃ | H, or COCH₃, or COCH₂CH₃ |
| 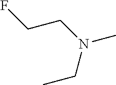 | CH₃ | H, or COCH₃, or COCH₂CH₃ |
| 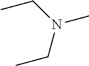 | CH₂CH₂F | H, or COCH₃, or COCH₂CH₃ |
| 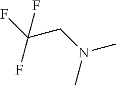 | CH₃ | H, or COCH₃, or COCH₂CH₃ |
| 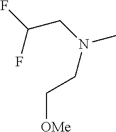 | CH₃ | H, or COCH₃, or COCH₂CH₃ |
| 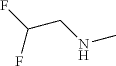 | CH₃ | H, or COCH₃, or COCH₂CH₃ |
| 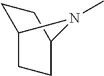 | CH₃ | H, or COCH₃, or COCH₂CH₃ |
| 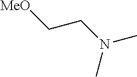 | CH₃ | H, or COCH₃, or COCH₂CH₃ |
| 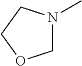 | CH₃ | H, or COCH₃, or COCH₂CH₃ |

Accordingly, it is particularly preferred that the compound of formula I is any one of the following compounds:

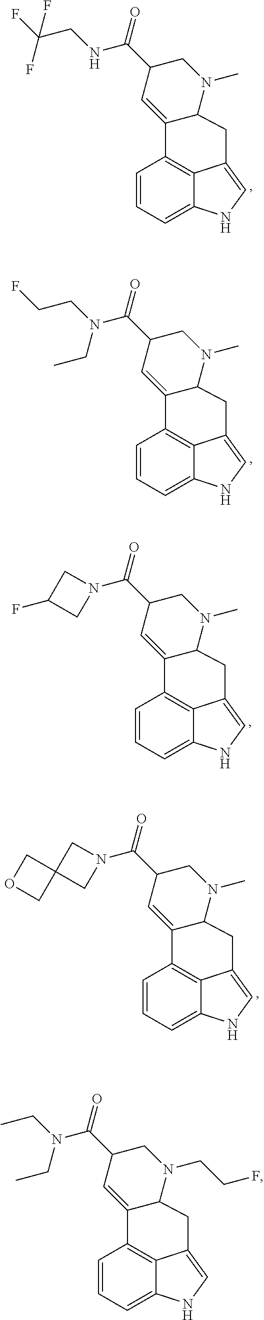

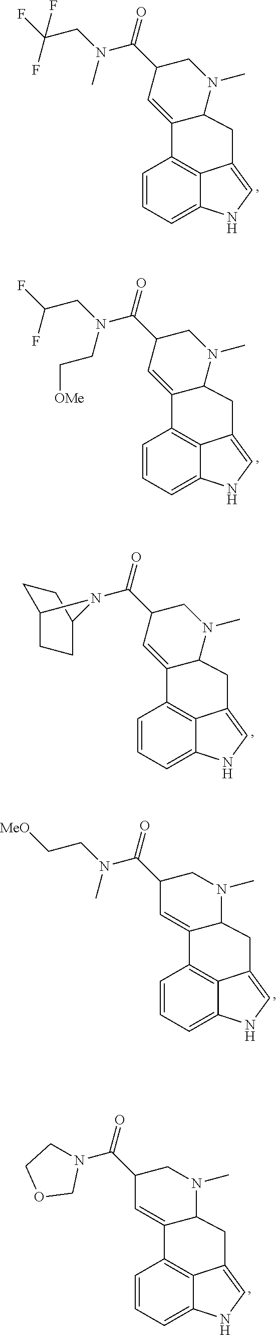

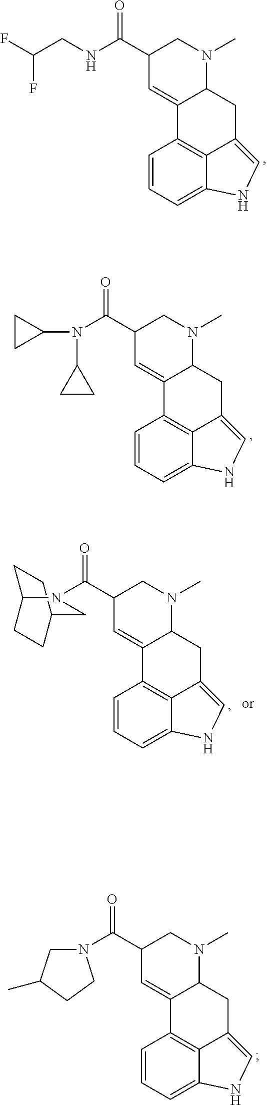

or a pharmaceutically acceptable salt of any one of the above-depicted compounds.

Moreover, it is preferred that the compounds of formula I (including any one of the specific exemplary compounds of formula I described herein) have the (5R,8R)-configuration, wherein the numbering is the same as that used for LSD. Accordingly, it is preferred that the compounds of formula I (including any one of the specific compounds described herein) have the following absolute configuration:

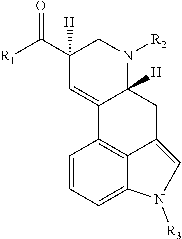

In a further aspect, the present invention provides the compounds having the following molecular structures as well as pharmaceutically acceptable salts thereof (in case of conflict between any chemical formula and the corresponding chemical name indicated herein, the present invention relates to both the compound defined by the chemical formula and the compound defined by the chemical name, and particularly relates to the compound defined by the chemical formula):

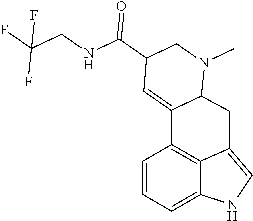 [SMILES: CN1CC(C(=O)NCC(F)(F)F)C=C2c3cccc4[nH]cc(c34)CC21]

lysergic acid mono-(trifluoroethyl)amide

Chemical formula: C₁₈H₁₈F₃N₃O

Molecular weight: 349.35

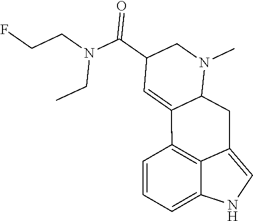 [SMILES: CCN(CCF)C(=O)C1C=C2c3cccc4[nH]cc(c34)CC2N(C)C1]

lysergic acid mono-fluorodiethylamide

Chemical formula: C₂₀H₂₄FN₃O

Molecular weight: 341.43

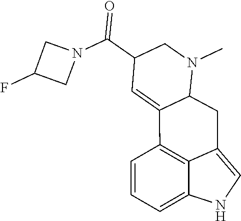 [SMILES: CN1CC(C(=O)N2CC(F)C2)C=C2c3cccc4[nH]cc(c34)CC21]

lysergic acid-3-fluoro-azetidide

Chemical formula: C₁₉H₂₀FN₃O

Molecular weight: 325.38

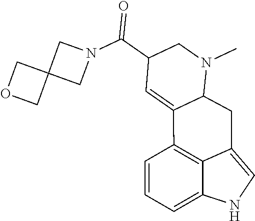 [SMILES: CN1CC(C(=O)N2CC3(COC3)C2)C=C2c3cccc4[nH]cc(c34)CC21]

2-oxa-6-azaspiro[3.3]heptyl-lysergic acid amide

Chemical formula: C₂₁H₂₃N₃O₂

Molecular weight: 349.43

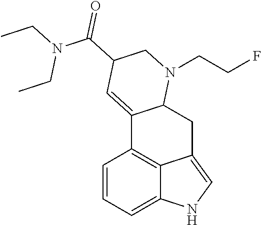 [SMILES: CCN(CC)C(=O)C1C=C2c3cccc4[nH]cc(c34)CC2N(CCF)C1]

6-(2-mono-fluoro-ethyl)-6-nor-lysergic acid diethylamide

Chemical formula: C₂₁H₂₆FN₃O

Molecular weight: 355.45

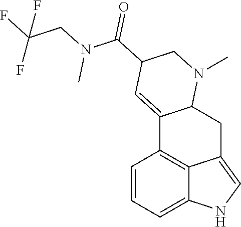 [SMILES: CN(CC(F)(F)F)C(=O)C1C=C2c3cccc4[nH]cc(c34)CC2N(C)C1]

lysergic acid methyl-(trifluoroethyl)amide

Chemical formula: C₁₉H₂₀F₃N₃O

Molecular weight: 363.38

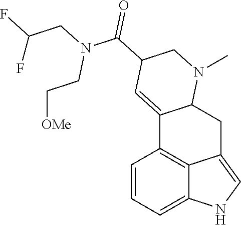 [SMILES: COCCN(CC(F)F)C(=O)C1C=C2c3cccc4[nH]cc(c34)CC2N(C)C1]

lysergic acid-(2,2-difluoroethyl)(2-methoxyethyl)amide

Chemical formula: C₂₁H₂₅F₂N₃O₂

Molecular weight: 389.45

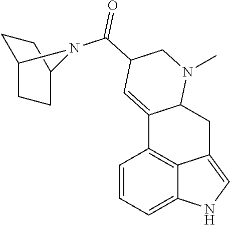 [SMILES: CN1CC(C(=O)N2C3CCC2CC3)C=C2c3cccc4[nH]cc(c34)CC21]

7-azabicyclo[2.2.1]heptyl-lysergic acid amide

Chemical formula: C₂₂H₂₅N₃O

Molecular weight: 347.46

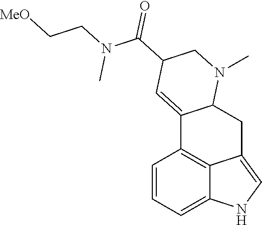 [SMILES: COCCN(C)C(=O)C1C=C2c3cccc4[nH]cc(c34)CC2N(C)C1]

lysergic acid-methyl-(2-methoxyethyl)amide

Chemical formula: C₂₀H₂₅N₃O₂

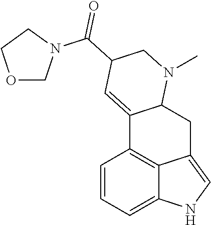 [SMILES: CN1CC(C(=O)N2CCOC2)C=C2c3cccc4[nH]cc(c34)CC21]

1,3-oxazolidinyl-lysergic acid amide

Chemical formula: C₁₉H₂₁N₃O₂

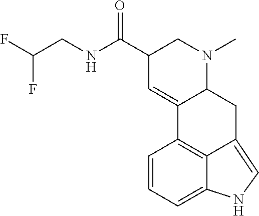 [SMILES: CN1CC(C(=O)NCC(F)F)C=C2c3cccc4[nH]cc(c34)CC21]

lysergic acid-mono-(2,2′-difluoroethyl)amide

Chemical formula: C₁₈H₂₁F₂N₃O

Molecular weight: 333.38

The present invention further provides a pharmaceutical/pharmacological composition comprising lysergic acid diethylamide (LSD) which is produced (or which is producible) by the method of production according to the invention, and optionally one or more pharmaceutically acceptable excipients. The present invention also provides a pharmaceutical/pharmacological composition comprising at least one LSD derivative, particularly at least one compound of formula I or a pharmaceutically acceptable salt thereof (which is preferably produced/producible by the method according to the invention), and optionally one or more pharmaceutically acceptable excipients. The invention likewise relates to the LSD produced by the method according to the invention, or the LSD derivatives provided herein (particularly a compound of formula I or a pharmaceutically acceptable salt thereof), or any of the aforementioned pharmaceutical compositions, for use in therapy (or for use as a medicament).

The invention further relates to the LSD produced by the method according to the invention (which may be present in non-salt form or in the form of a pharmaceutically acceptable salt), or an LSD derivative provided herein (particularly a compound of formula I, which may be present in non-salt form or in the form of a pharmaceutically acceptable salt), or a pharmaceutical composition comprising any of the aforementioned entities, for use in the treatment of a serotonin 5-HT_{2A} receptor associated disease/disorder. In particular, the invention relates to the LSD produced by the method according to the invention (which may be present in non-salt form or in the form of a pharmaceutically acceptable salt), or an LSD derivative (particularly a compound of formula I, which may be present in non-salt form or in the form of a pharmaceutically acceptable salt), or a pharmaceutical composition comprising any of the aforementioned entities, for use in the treatment of an anxiety disorder, attention deficit hyperactivity disorder (ADHD), depression, cluster headache, a condition associated with cancer, diminished drive, burn-out, bore-out, migraine, pulmonary hypertension, schizophrenia, an eating disorder, Parkinson's disease, dementia, nausea, or vomiting.

The invention also refers to the use of the LSD produced by the method according to the invention or the use of an LSD derivative provided herein (particularly a compound of formula I or a pharmaceutically acceptable salt thereof) in the manufacture of a medicament for the treatment of a serotonin 5-HT_{2A} receptor associated disease/disorder, preferably for the treatment of an anxiety disorder, attention deficit hyperactivity disorder (ADHD), depression, cluster headache, a condition associated with cancer, diminished drive, burn-out, bore-out, migraine, pulmonary hypertension, schizophrenia, an eating disorder, Parkinson's disease, dementia, nausea, or vomiting.

Moreover, the invention provides a method of treating a disease/disorder, particularly a serotonin 5-HT_{2A} receptor associated disease/disorder, in a subject in need thereof, the method comprising administering a therapeutically effective amount of the LSD produced by the method according to the invention or of an LSD derivative provided herein (particularly a compound of formula I or a pharmaceutically acceptable salt thereof) to the invention to said subject. It is preferred that the disease/disorder to be treated is an anxiety disorder, attention deficit hyperactivity disorder (ADHD), depression, cluster headache, a condition associated with cancer, diminished drive, burn-out, bore-out, migraine, pulmonary hypertension, schizophrenia, an eating disorder, Parkinson's disease, dementia, nausea, or vomiting.

Thus, the lysergic acid diethylamide (LSD) produced according to the method of the invention in high yield and/or high purity or the lysergic acid diethylamide derivatives of the invention, in particular those also produced in high yield and/or high purity, are suitable for the treatment of anxiety disorders, ADHD, depression, cluster headache, cancer-associated conditions, diminished drive, burn-out, bore-out, migraine, pulmonary hypertension, schizophrenia, an eating disorder, Parkinson's disease, dementia, nausea, and vomiting, and other diseases resulting from disturbances of signal transduction at the serotonin 5-HT_{2A} receptor.

As such, the compounds of the invention may be administered as part of a pharmacological formulation, preferably by “microdosing”, i.e. by administering small doses, which do not elicit hallucinations in the amounts used, and the dosage interval of which is in the range of days or even weeks.

The following definitions apply throughout the present specification and the claims, unless specifically indicated otherwise.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “C₀ alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 1H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, p-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, morpholinyl, pyrrolidinyl, or tetrahydrofuranyl.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “C₃₋₇ cycloalkyl” refers to a monocyclic saturated hydrocarbon ring group having 3 to 7 ring members (i.e., 3 to 7 carbon ring atoms). Corresponding examples include cyclopropyl, cyclobutyl, cyclopentyl, cyclohexyl, or cycloheptyl. Unless defined otherwise, a particularly preferred “C₃₋₇ cycloalkyl” is cyclopropyl.

As used herein, the term “halogen” (or its plural form “halogens”) refers to fluoro (—F), chloro (—Cl), bromo (—Br), or iodo (—I). It will be understood that if a compound or a chemical group is substituted with “halogens” (e.g., two or more “halogens”), the corresponding halogen atoms may be the same or different, e.g., they may all be fluoro or they may be selected independently from fluoro, chloro, bromo, and iodo.

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CH₂F, —CH₂—CF₂—CF₃, or —CH(CF₃)₂. A preferred “haloalkyl” group is fluoroalkyl. A particularly preferred “haloalkyl” group is —CH₂CH₂F.

As used herein, the term “fluoroalkyl” refers to an alkyl group substituted with one or more (preferably 1 to 6, more preferably 1 to 3) fluoro atoms (—F). It will be understood that the maximum number of fluoro atoms is limited by the number of available attachment sites and, thus, depends on the number of carbon atoms comprised in the alkyl moiety of the fluoroalkyl group. “Fluoroalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CH₂F, —CH₂—CF₂—CF₃, or —CH(CF₃)₂. A particularly preferred “fluoroalkyl” group is —CH₂CH₂F.

The terms “bond” and “covalent bond” are used herein synonymously, unless explicitly indicated otherwise or contradicted by context.

As used herein, the term “amino acid” refers, in particular, to any one of the 20 standard proteinogenic α-amino acids (i.e., Ala, Arg, Asn, Asp, Cys, Glu, Gln, Gly, His, lie, Leu, Lys, Met, Phe, Pro, Ser, Thr, Trp, Tyr, or Val) but also to any non-proteinogenic and/or non-standard α-amino acid (such as, e.g., ornithine, citrulline, homolysine, pyrrolysine, 4-hydroxyproline, α-methylalanine (i.e., 2-aminoisobutyric acid), norvaline, norleucine, terleucine (i.e., tert-leucine), labionin, or an alanine or glycine that is substituted at the side chain with a cyclic group such as, e.g., cyclopentylalanine, cyclohexylalanine, phenylalanine, naphthylalanine, pyridylalanine, thienylalanine, cyclohexylglycine, or phenylglycine), any β-amino acid (e.g., β-alanine), any γ-amino acid (e.g., γ-aminobutyric acid, isoglutamine, or statine), any δ-amino acid, and/or any other compound comprising at least one carboxylic acid group and at least one amino group. Unless defined otherwise, an “amino acid” preferably refers to an α-amino acid, more preferably to any one of the 20 standard proteinogenic α-amino acids (which may be present as the L-isomer or the D-isomer, and are preferably present as the L-isomer). It will be understood that if an “amino acid is attached via a —CO— group formed from a carboxylic acid group of the amino acid”, this means that one carboxylic acid group of the corresponding amino acid is present in the form of a —CO— group and the amino acid is attached to the remainder of the compound via this —CO— group; for example, if an alanine is attached via a —CO— group formed from a carboxylic acid group of said alanine, then the resulting moiety is —CO—CH(—CH₃)—NH₂.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula I can be interpreted as referring to a composition comprising “one or more” compounds of formula I.

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

In principle, the order of the different steps of any method described herein can be chosen as desired, i.e., the method steps can be followed in the indicated order or in a different order. It will be understood that some method steps may build upon one or more prior method steps and may thus require that the corresponding prior mentioned method step(s) must be conducted first. For each method described herein, it is preferred that the corresponding method steps are conducted in the specific order in which they are listed.

As used herein, the term “about” preferably refers to ±10% of the indicated numerical value, more preferably to ±5% of the indicated numerical value, and in particular to the exact numerical value indicated.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

Unless specifically indicated otherwise, all properties and parameters referred to herein (including, e.g., any pH values) are preferably to be determined at standard ambient temperature and pressure conditions, particularly at a temperature of 25° C. (298.15 K) and at an absolute pressure of 101.325 kPa (1 atm).

The term “d” (i.e., lowercase D), when used in connection with the chemical name of an optically active compound (such as, e.g., “d-lysergic acid diethylamide” or “d-LSD”), indicates that the corresponding compound is dextrorotary. In accordance with established chemical terminology, the term “d” is synonymous with “(+)”, i.e., it designates the (+)-stereoisomer of the respective compound. A term such as “d-LSD” is thus synonymous with “(+)-LSD”.

The term “I” (i.e., lowercase L), when used in connection with the chemical name of an optically active compound, indicates that the corresponding compound is levorotary. In accordance with established chemical terminology, the term “I” is synonymous with “(−)”, i.e., it designates the (−)-stereoisomer of the respective compound.

The present invention relates to the lysergic acid diethylamide (LSD) and the lysergic acid diethylamide (LSD) derivatives provided herein, including the compounds of formula I, in any form, e.g., in non-salt form or in the form of a salt, particularly a pharmaceutically acceptable salt.

The scope of the present invention thus embraces all pharmaceutically acceptable salt forms of LSD or of the LSD derivatives of formula I which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Further pharmaceutically acceptable salts are described in the literature, e.g., in Stahl P H & Wermuth C G (eds.), “Handbook of Pharmaceutical Salts: Properties, Selection, and Use”, Wiley-VCH, 2002 and in the references cited therein. Preferred examples of a pharmaceutically acceptable salt of the LSD produced according to the invention or of the LSD derivatives of formula I include, in particular, a tartrate salt, a fumarate salt, an oxalate salt, or a maleate salt.

The scope of the present invention also embraces the LSD or the LSD derivatives provided herein in any hydrated or solvated form, and in any physical form, including any amorphous or crystalline forms.

Moreover, the LSD or the LSD derivatives of formula I may exist in the form of different isomers, in particular stereoisomers (e.g., enantiomers or diastereomers). All such isomers are contemplated as being part of the present invention, either in admixture or in pure or substantially pure form. Any tautomers of the compounds described herein are also embraced by the present invention. As for stereoisomers, the invention embraces the isolated optical isomers of the LSD derivatives according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers may also be prepared by using corresponding optically active starting materials in their synthesis, or they may be obtained from corresponding racemates via salt formation with an optically active acid followed by crystallization.

The LSD produced/producible by the method according to the present invention as well as the LSD derivatives provided herein may be administered as compounds perse or may be formulated as pharmaceutical/pharmacological compositions or medicaments. The pharmaceutical compositions/medicaments may optionally comprise one or more pharmaceutically acceptable excipients, such as carriers, diluents, fillers, disintegrants, lubricating agents, binders, colorants, pigments, stabilizers, preservatives, and/or antioxidants.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

In principle, the LSD or the LSD derivatives of formula I or the corresponding pharmaceutical compositions may be administered to a subject by any convenient route of administration. Various routes for administering pharmaceutical agents are known in the art and include, inter alia, oral (e.g., as a tablet, capsule, ovule, elixir, or as an ingestible solution or suspension), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

It is preferred that the LSD or the LSD derivatives according to the invention (or corresponding pharmaceutical compositions) are administered orally. Suitable dosage forms for oral administration include, e.g., coated or uncoated tablets, soft gelatin capsules, hard gelatin capsules, lozenges, troches, solutions, emulsions, suspensions, syrups, elixirs, powders or granules for reconstitution, dispersible powders or granules, medicated gums, chewing tablets, or effervescent tablets.

As a further preferred route of administration, the LSD or the LSD derivatives according to the invention (or corresponding pharmaceutical compositions) may be administered parenterally, particularly intravenously (e.g., by intravenous injection). For parenteral administration, the LSD or the LSD derivatives can be used, e.g., in the form of a sterile aqueous solution which may contain other substances, for example, sufficient salts or glucose to make the solution isotonic with blood. The aqueous solution may be suitably buffered, preferably to a physiological pH. The preparation of suitable parenteral formulations under sterile conditions is readily accomplished by standard pharmaceutical techniques known in the art.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal. Most preferably, the subject/patient to be treated in accordance with the invention is a human.

As used herein, the term “treatment” (or “treating”) in relation to a disease or disorder refers to the management and care of a patient for the purpose of combating the disease or disorder, such as to reverse, alleviate, inhibit or delay the disease or disorder, or one or more symptoms of such disease or disorder. It also refers to the administration of a compound or a composition for the purpose of preventing the onset of symptoms of the disease or disorder, alleviating such symptoms, or eliminating the disease or disorder. Preferably, the “treatment” is curative, ameliorating or palliative.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula I. Likewise, the invention specifically relates to each combination of features/embodiments for the different steps of each method described herein.

In this specification, a number of documents including patent applications/patents and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

### BRIEF DESCRIPTION OF THE FIGURES

FIG. 1: A sample of the reaction mixture of Example 1 shows the following components in LC/MS (liquid chromatography-mass spectrometry) at a wavelength of 312 nm: 0.85% lysergic acid; 69.4% LSD; 29.7% iso-LSD.

FIG. 2: TLC (thin layer chromatography) evaluation of fractions 1-10 of the reaction product following column chromatographic purification (see Example 3).

FIG. 3: TLC evaluation of fractions 10-13 of the reaction product following column chromatographic purification (see Example 3).

FIG. 4: Production of lysergic acid-mono-(2,2,2-trifluoroethyl)amide (see Example 5), TLC shows conversion after 24 h.

FIG. 5: TLC evaluation of the production of lysergic acid-mono-(2,2,2-trifluoroethyl)amide following purification. According to LC-MS, fractions 1-7 contain 99% pure product.

The invention will now be described by reference to the following examples which are merely illustrative and are not to be construed as a limitation of the scope of the present invention.

### EXAMPLES

### Example 1: Method of Production of Lysergic Acid Diethylamide (LSD)

Lysergic acid hydrate (10 mmol/2.86 g) is suspended in ethyl acetate (200 ml) at 25° C.

Diethylamine (100 mmol/10.4 ml) is added and aerated with argon. This results in a whitish-gray suspension.

A 50% (wt %) solution of propane-phosphonic acid anhydride (T3P) in ethyl acetate (30 mmol/19.08 g) is added dropwise through the septum during 70 min.

During the addition, the initial suspension already starts to turn clear at less than 2 ml propane-phosphonic acid anhydride solution, and a few minutes after the addition, a clear solution is obtained. Thereby, a slightly warm shading can be observed.

The immediate dissolution of the otherwise not easily dissoluble lysergic acid may be explained by the formation of intermediary lysergic acid ethyl ester, which is subsequently detectable in the crude product as traces. Stirring is continued for further 2.5 h under argon at 26° C.

A sample of the reaction mixture shows the following components in LC/MS at a wavelength of 312 nm: 0.85% lysergic acid; 69.4% LSD; 29.7% iso-LSD (see also FIG. 1).

The reaction is stirred for further 3 h at 26° C. under argon, followed by dilution with ethyl acetate (200 ml) to stop the reaction. The dark yellow slightly colloidal reaction solution is extracted with 150 ml water. The aqueous phase has a pH of 9.

The organic phase is dried over little MgSO₄ and concentrated at 45° C. at up to 40 mbar.

This yields 4.1 g of a yellow-brown oil (crude product).

HPLC at 312 nm shows 99.9% of product (both isomers) and 0.1% of ethyl ester.

### Example 2: Isomer Optimization

The complete amount is dissolved in absolute ethanol (60 ml). Sodium methoxide (520 mg) (0.95 eq) is added and stirred for 3 h at 50° C.

After two hours, sampling for HPLC yielded 82% of LSD.

After 3.5 hours, sampling for HPLC yielded 84%.

After 4 hours, the reaction was stopped. At this time, the isomer ratio according to HPLC at 312 nm has changed to 84%:16% of LSD to iso-LSD.

The product is diluted with water (100 ml) and rotated in (i.e. the water is distilled off on a rotary evaporator) at pH 11-11.5. The aqueous residue is again diluted with water (100 ml) and extracted four times with 150 ml ethyl acetate.

The product is dried over magnesium sulfate and then concentrated.

This yields 3.8 g of a brown oil.

Other concentrations of NaOMe and different times were tried as well.

The equilibrium of >80% LSD to iso-LSD had adjusted after 2-3 h. Even after 12 h, this ratio had not significantly improved. However, signs of decomposition could already be seen after 12-24 hours.

### Example 3: Column Chromatographic Purification

Isomer-optimized crude material is column treated over 300 g silica using the eluent mixture toluene/ethanol at 95:5.

Crude product is dissolved in toluene/ethanol at 70:30 (15 ml) and applied.

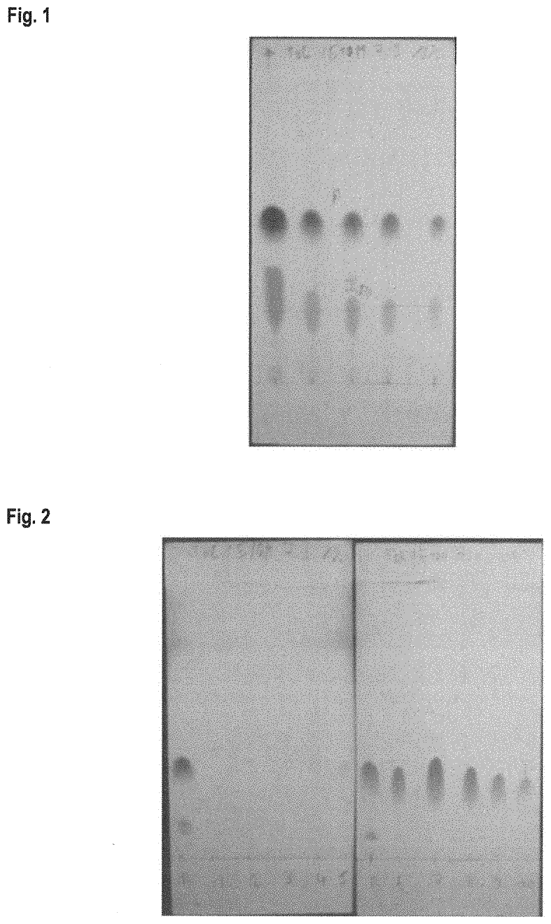

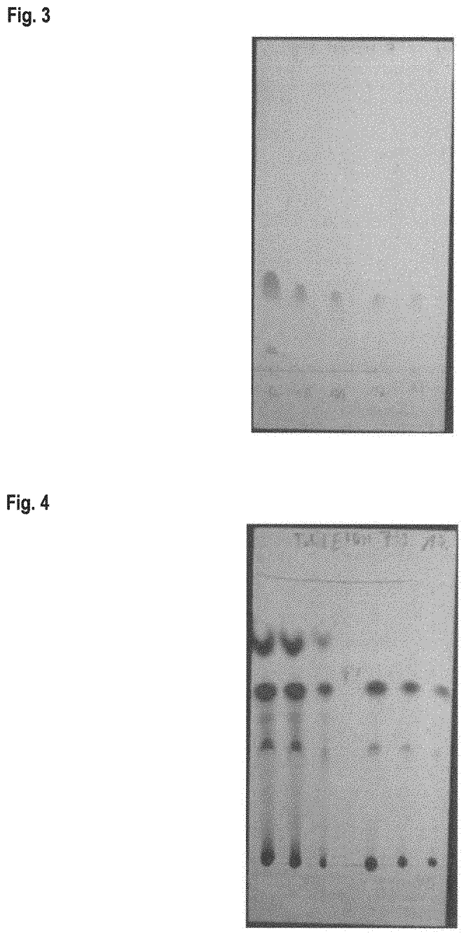

The column is started with toluene/ethanol at 95:5. Following one liter of this eluent it is changed to toluene/ethanol at 90:10. One of the impurities runs first as a green band (365 nm). The product runs directly behind it as a bright violet region (365 nm) (see FIGS. 2 and 3).

The product fractions are concentrated, on a rotary evaporator. Thereby, 2.9 g of a bright foam is obtained. This corresponds to 89% of the theoretical yield.

HPLC at 312 nm confirms a purity of more than 99%. No iso-LSD at all is present anymore.

### Example 4: Production of LSD Derivatives

The novel LSD derivatives according to the present invention can be produced using the method of production of the invention in high purity and yield.

For this purpose, a person skilled in the art merely has to select the respective amine compound (in part as a hydrochloride) accordingly. Instead of diethylamine the person skilled in the art has to use 10 eq amine HCl+10 eq DiPEA. The amine to be reacted is produced in situ in this way.

The purity to be expected is more than 95% and the yield is at least 50%.

### Example 5: Alternative Production of Lysergic Acid-Mono-(2,2,2-Trifluoroethyl)Amide

In order to demonstrate the producibility of the LSD derivatives with conventional methods as well, a few derivatives were synthesized by using conventional methods of production. The compound is producible, but the yields are significantly lower than by using the method of the invention.

Lysergic acid hydrate (11 mmol; 2.94 g) is suspended in dichloromethane (200 ml) at 26° C. and is aerated with argon. Diisopropylethylamine (11 mmol; 1.9 ml) is added dropwise through the septum.

Then, carbodiimidazole (17 mmol; 2.75 mg) is colloidally dissolved in dichloromethane (60 ml) and added dropwise through the septum.

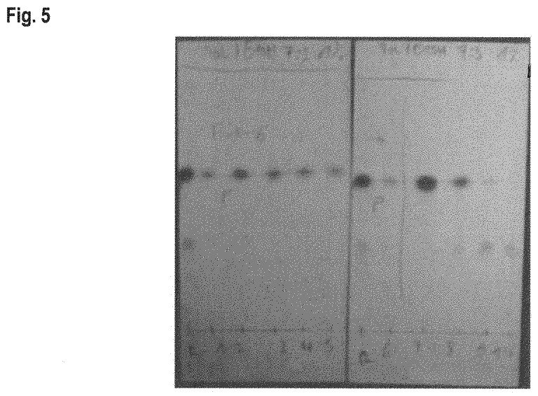

This is stirred for 20 min at 26° C. Then, 2,2,2-trifluoroethylamine base (22 mmol; 1.73 ml) is added dropwise through the septum and stirred for 24 h at 26° C. TLC shows a conversion after 24 h (see FIG. 4).

On the next day, the reaction is stopped with 2% ammonia solution (30 ml) and the reaction mixture is diluted with dichloromethane (100 ml). Then, the organic phase is separated and washed with water (100 ml) and saturated saline (100 ml). Thereafter, it is dried over magnesium sulfate and concentrated on the rotary evaporator.

Thereby, 2.5 g of a brown oil is obtained.

The purification is done by means of a 150 g silica column using toluene/ethanol at 95:5 as an eluent.

The crude product is dissolved in toluene/dichloromethane at 1:1 (10 ml) and applied. The column is started with pure toluene (300 ml). Thereafter, the eluent is changed to toluene/ethanol at 95:5. According to LC-MS, fractions 1-7 contain 99% pure product (see FIG. 5).

Without previous isomer optimization, about 0.7 g of a brown oil is obtained. This corresponds to a yield of 18%.

### Example 6: Alternative Production of 2-oxa-6-azaspiro[3.3]heptyl-lysergic acid amide

In order to demonstrate the producibility of the LSD derivatives with conventional methods as well, a few derivatives were synthesized by using conventional methods of production. The compound is producible, but the yields are significantly lower than by using the method of the invention.

Lysergic acid hydrate (3 mmol; 858 mg) is suspended in chloroform (40 ml) at 26° C. and is aerated with argon.

Diisopropylethylamine (1.5 mmol; 0.26 ml) is added dropwise through the septum.

Then, carbodiimidazole (6 mmol; 972 mg) is colloidally dissolved in chloroform (11 ml) and added dropwise through the septum.

This is stirred for 35 min at 26° C. Then, 2-oxa-6-azaspiro[3.3]heptane base (6 mmol; 594 mg) is added dropwise through the septum and stirred at 26° C.

After stirring for 2.5 h at 26° C., the formation of a product can be observed by TLC/LC-MS (see FIG. 5).

### Example 7: Conversion into 2-oxa-6-azaspiro[3.3]heptyl-lysergic acid amide and iso-2-oxa-6-azaspiro[3.3]heptyl-lysergic acid amide

Conversion into 58% 2-oxa-6-azaspiro[3.3]heptyl-lysergic acid amide and iso-2-oxa-6-azaspiro[3.3]heptyl-lysergic acid amide.

At 312 nm, 31% of lysergic acid are still measurable. Therefore, stirring at 26° C. is continued for further 18 h.

After 2.5 h, the reaction mixture contains 58% isomer mixture and 31% starting material.

Then, the reaction mixture is diluted with chloroform (100 ml) and extracted with water (40 ml).

The organic phase is dried over magnesium sulfate and concentrated on the rotary evaporator.

1.10 g of a yellow-brown oil is obtained, which still contains amounts of diisopropylethylamine.

The purification is done by means of a 100 g silica column using dichloromethane/methanol at 80:20 as an eluent. The crude product is dissolved in pure dichloromethane (8 ml) and applied. The column is run isocratically with dichloromethane/methanol at 80:20 as an eluent.

According to HPLC at 312 nm, fractions 3 and 4 contain 2-oxa-6-azaspiro[3.3]heptyl-lysergic acid amide at a purity of 97%. Fractions 5-9 contain both isomers at a ratio of 1:1.

Without previous isomer optimization, 0.32 g of a yellow oil is thus obtained.

This corresponds to a yield of 31%.

### Example 8: Binding Inhibition by the LSD Derivatives of the Invention at Multiple Receptor Targets

### Introduction

Four novel LSD derivatives according to the present invention (referred to as “Compound 1”, “Compound 2”, “Compound 3”, and “Compound 4”; structures depicted below) were screened at 10 μM at key receptors of interest in order to determine % binding inhibition. Targets were selected due to prior evidence that LSD binds these targets with moderate or high affinities in vitro, as well as their functional significance in the context of human health.

Generally, % inhibition results greater than 50% are interpreted as displaying a potentially significant interaction.

### Compound 1 (2-oxa-6-azaspiro[3.3]heptyl-lysergic acid amide)

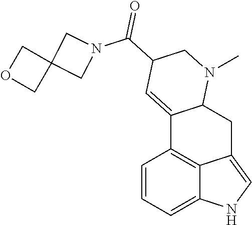 [SMILES: CN1CC(C(=O)N2CC3(COC3)C2)C=C2c3cccc4[nH]cc(c34)CC21]

### Compound 2 (6-(2-mono-fluoro-ethyl)-6-nor-lysergic acid diethylamide)

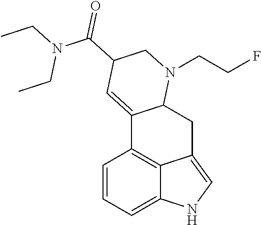 [SMILES: CCN(CC)C(=O)C1C=C2c3cccc4[nH]cc(c34)CC2N(CCF)C1]

### Compound 3 (lysergic acid mono-(trifluoroethyl)amide)

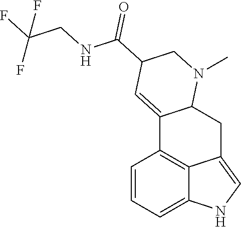 [SMILES: CN1CC(C(=O)NCC(F)(F)F)C=C2c3cccc4[nH]cc(c34)CC21]

### Compound 4 (lysergic acid monofluoro diethylamide)

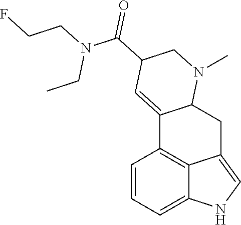 [SMILES: CCN(CCF)C(=O)C1C=C2c3cccc4[nH]cc(c34)CC2N(C)C1]

### Methods

### 5-HT_{2A} Receptor

Human recombinant serotonin 5-HT_{2A} receptors expressed in CHO-K1 cells were used in modified Tris-HCl buffer pH 7.4. A 30 μg aliquot of membrane protein was incubated with 0.5 nM [³H]Ketanserin for 60 minutes at 25° C. Non-specific binding was estimated in the presence of 1 μM Mianserin. Receptors were filtered and washed, the filters were then counted to determine [³H]Ketanserin specifically bound. Test compounds were screened at 10 μM.

### 5-HT_{2B} Receptor

Human recombinant serotonin 5-HT_{2B} receptor expressed in CHO-K1 cells were used to prepare membranes in modified Tris-HCl buffer pH 7.4. A 30 μg aliquot of membrane protein was incubated with 1.2 nM [³H]LSD for 60 minutes at 37° C. Non-specific binding was estimated in the presence of 10 μM serotonin. Membranes were filtered and washed, the filters were then counted to determine [³H]LSD specifically bound. Test compounds were screened at 10 μM.

### 5-HT_{2C} Receptor

CHO-K1 cells stably transfected with a plasmid encoding the human recombinant serotonin 5-HT₂c receptors were used to prepare membranes in modified Tris-HCl buffer pH 7.4. A 3.2 μg aliquot of membrane protein was incubated with 1.0 nM [³H]Mesulergine for 60 minutes at 25° C. Non-specific binding was estimated in the presence of 1 μM Mianserin. Membranes were filtered and washed, the filters were then counted to determine [³H]Mesulergine specifically bound. Test compounds were screened at 10 μM.

### 5-HT_{1A} Receptor

Human recombinant serotonin 5-HT_{1A} receptors expressed in CHO-K1 cells were used in modified Tris-HCl buffer pH 7.4. An 8 μg aliquot of membrane protein was incubated with 1.5 nM [³H]8-OH-DPAT for 60 minutes at 25° C. Non-specific binding was estimated in the presence of 10 μM metergoline. Receptors were filtered and washed, the filters were then counted to determine [³H]8-OH-DPAT specifically bound. Test compounds were screened at 10 μM.

### D₁ Receptor

Human recombinant dopamine D₁ receptors expressed in CHO cells were used in modified Tris-HCl buffer pH 7.4. A 20 μg aliquot was incubated with 1.4 nM [³H]SCH-23390 for 120 minutes at 37° C. Non-specific binding was estimated in the presence of 10 μM (+)-butaclamol. Receptors were filtered and washed, the filters were then counted to determine [³H]SCH-23390 specifically bound. Test compounds were screened at 10 μM.

### D_{2S} Receptor

Human recombinant dopamine D_{2S} receptors expressed in CHO cells were used in modified Tris-HCl buffer pH 7.4. A 15 μg aliquot was incubated with 0.16 nM [³H]Spiperone for 120 minutes at 25° C. Non-specific binding was estimated in the presence of 10 μM haloperidol. Receptors were filtered and washed, the filters were then counted to determine [³H]Spiperone specifically bound. Test compounds were screened at 10 μM.

### D_{2L} Receptor

Human recombinant dopamine D_{2L} receptor expressed in CHO cells were used in modified Tris-HCl buffer pH 7.4. A 20 μg aliquot was incubated with 0.16 nM [³H]Spiperone for 120 minutes at 25° C. Non-specific binding was estimated in the presence of 10 μM haloperidol. Receptor proteins were filtered and washed, the filters were then counted to determine [³H]Spiperone specifically bound. Test compounds were screened at 10 μM.

### D₃ Receptor

Human recombinant dopamine D₃ receptors expressed in CHO cells were used in modified Tris-HCl buffer pH 7.4. A 10 μg aliquot was incubated with 0.7 nM [³H]Spiperone for 120 minutes at 37° C. Non-specific binding was estimated in the presence of 25 μM S(−)-sulpiride. Receptors were filtered and washed, the filters were then counted to determine [³H]Spiperone specifically bound. Test compounds were screened at 10 μM.

### D₄₇ Receptor

CHO-K1 cells stably transfected with a plasmid encoding the human dopamine D_{4.7} receptor were used to prepare membranes in modified Tris-HCl buffer pH 7.4. A 60 μg aliquot of membrane was incubated with 1.5 nM [³H]Spiperone for 120 minutes at 25° C. Non-specific binding was estimated in the presence of 10 μM haloperidol. Membranes were filtered and washed, the filters were then counted to determine [³H]Spiperone specifically bound. Test compounds were screened at 10 μM.

### A_{2A} Receptor

Human recombinant adenosine A_{2A} receptors expressed in human HEK-293 cells were used in modified Tris-HCl buffer pH 7.4. A 15 μg aliquot was incubated with 50 nM [³H]CGS-21680 for 90 minutes at 25° C. Non-specific binding was estimated in the presence of 50 μM NECA. Receptor were filtered and washed, the filters were then counted to determine [³H]CGS-21680 specifically bound. Test compounds were screened at 10 μM.

### Analysis

Experimental results were expressed as a percent of control specific binding, via the equation:

(100−(measured specific binding×100)

$$( 100 - ( \frac{measured specific binding}{control specific binding} \times 100 )$$

For each target, data from two experimental replicates were averaged to generate a mean % inhibition.

### Results

The experimental data obtained are summarized in the following Table 1.

TABLE 1

| | Compound 1 | Compound 2 | Compound 3 | Compound 4 |
|---|---|---|---|---|
| Target | % inhibition | % inhibition | % inhibition | % inhibition |
| 5-HT_{2A} | 95.3 | 95.7 | 93.1 | 97.1 |
| 5-HT_{2B} | 96.8 | 90.5 | 94.9 | 97.9 |
| 5-HT_{2C} | 85.8 | 4.8 | 89.4 | 100.4 |
| 5-HT_{1A} | 96.3 | 100.2 | 100.6 | 100.5 |
| D_{2S} | 83.1 | 0.6 | 64.2 | 92.1 |
| D_{2L} | 78.8 | 7.9 | 63.1 | 92.7 |
| D₁ | 85.6 | 4.3 | 53.8 | 97.1 |
| D₃ | 94.5 | 14.2 | 94.7 | 97.7 |
| D_{4.7} | 80.9 | 2.8 | 20.1 | 71.6 |
| A_{2A} | −12.3 | −2.5 | −2.0 | 12.3 |

% inhibition of novel compounds at target receptors of interest. Higher % inhibition indicates a likely stronger ligand-receptor interaction.

These results demonstrate that the LSD derivatives according to the present invention, including Compound 1, Compound 2, Compound 3 and Compound 4, are advantageously selective ligands of the 5-HT_{2A} receptor.

Moreover, compounds such as Compound 2 have been found to exhibit an advantageous receptor subtype selectivity. Thus, a very high % inhibition was observed for Compound 2 at the 5-HT_{2A} and 5-HT_{1A} receptors while no significant interaction was determined at the 5-HT_{2C}, A_{2A} or any dopamine receptors assayed. For Compound 3, significant interaction was observed at all 5-HT targets while no significant interaction was observed at the A_{2A} or D₄₇ receptors; the interaction with D₃ showed a higher % inhibition than at other dopamine receptors, which may indicate a greater selectivity for D₃ over other dopamine receptors. For all tested compounds, including Compound 1 and Compound 4, no significant interaction was determined at the A_{2A} receptor.

These findings confirm that the LSD derivatives provided herein are particularly well suited for use in therapy, including for the treatment of serotonin 5-HT_{2A} receptor associated diseases/disorders.

### Example 9: Metabolization Study Using a Pooled Human Liver Microsome Assay

Pooled human liver microsome (pHLM) samples were prepared by adding novel LSD derivatives (“L1”, “L2”, “L3” or “L4”; structures shown in the schemes below) (approx. 1 mg/mL in ACN) solution to a reaction mixture (approx. 10 μg/mL final substrate concentration) consisting of pHLM, phosphate buffer, and deionized water. Incubation was performed for 30 minutes at 37° C. and stopped by the addition of ice-cold ACN. After centrifugation, the supernatant was diluted (1:10 for liquid chromatography-electrospray ionization-tandem mass spectrometry (LC-ESI-MS/MS) and 1:2 for liquid chromatography-electrospray ionization quadrupole time-of-flight mass spectrometry (LC-ESI-QToF-MS) analysis with mobile phase A/B (50/50, v/v). Two negative control samples were processed the same way, one with 2.5 μL phosphate buffer instead of pHLM and the second with 0.5 μL ACN instead of the substrate.

For the identification of tentative main metabolites, a Nexera X2 UHPLC (Shimadzu, Duisburg, Germany) coupled to a QTRAP® 5500 triple quadrupole linear ion trap mass spectrometer (SCIEX, Darmstadt, Germany) was utilized for analysis of pHLM. Phase I metabolism of the four novel LSD derivatives as well as a corresponding measurement of LSD as a reference were conducted.

As a result, for all four LSD derivatives similar metabolism derivatization reactions like hydroxylation, dihydroxylation, amine-demethylation and dealkylation of the amide moieties in analogy to the metabolism of LSD have been observed, as illustrated in the following schemes:

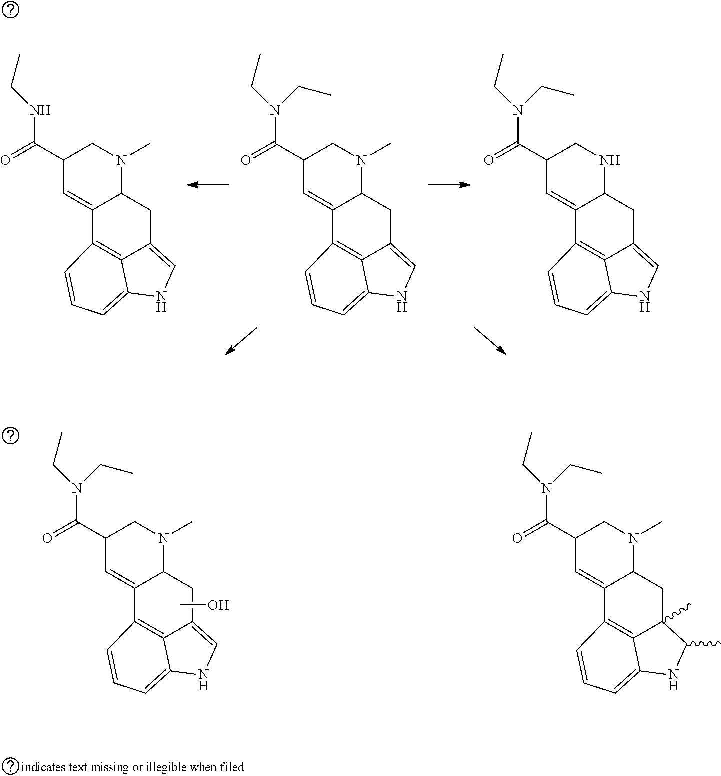

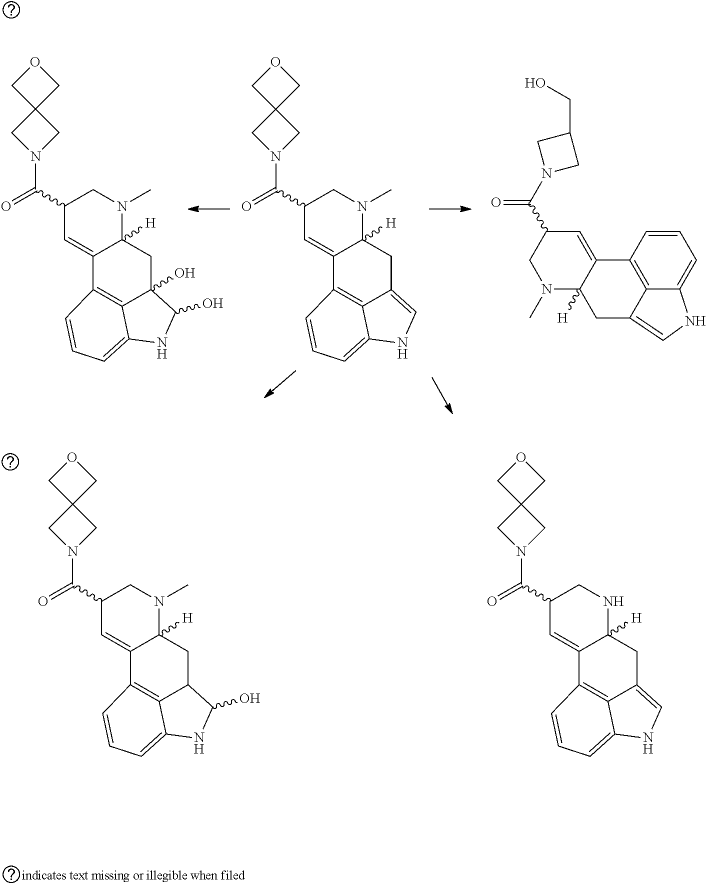 [SMILES: CN1CC(C(=O)N2CC3(COC3)C2)C=C2c3cccc4c3C(O)(CC21)C(O)N4 ; CN1CC(C(=O)N2CC3(COC3)C2)C=C2c3cccc4[nH]cc(c34)CC21 ; CN1CC(C(=O)N2CC(CO)C2)C=C2c3cccc4[nH]cc(c34)CC21 ; CN1CC(C(=O)N2CC3(COC3)C2)C=C2c3cccc4c3C(CC21)C(O)N4 ; O=C(C1C=C2c3cccc4[nH]cc(c34)CC2NC1)N1CC2(COC2)C1]

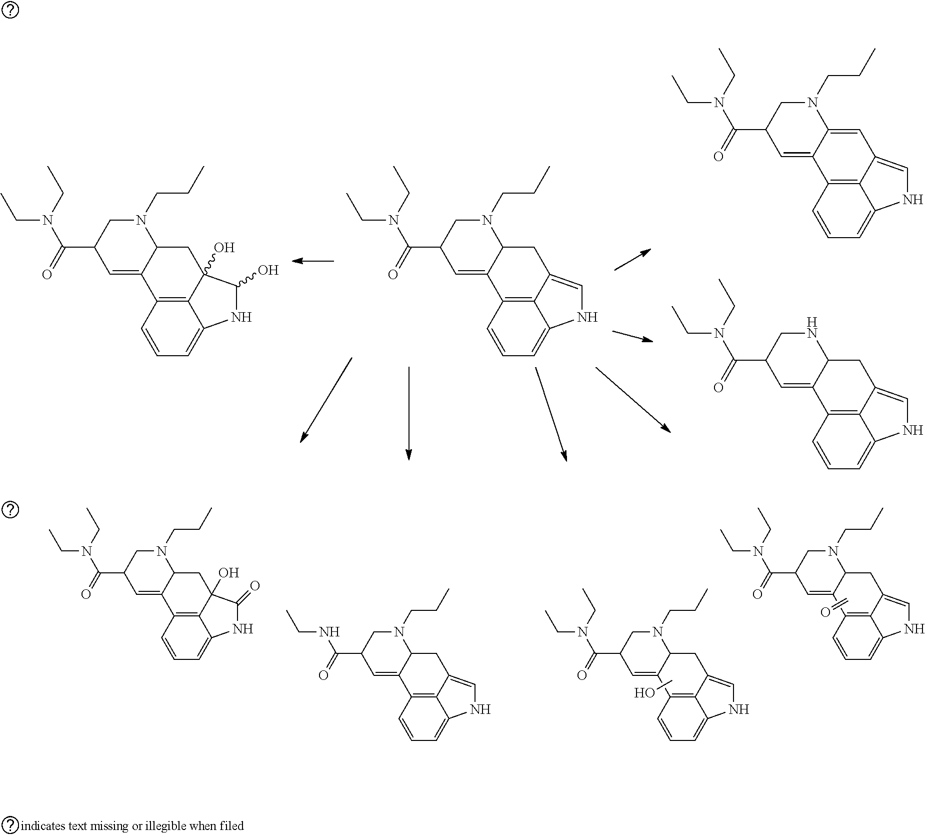

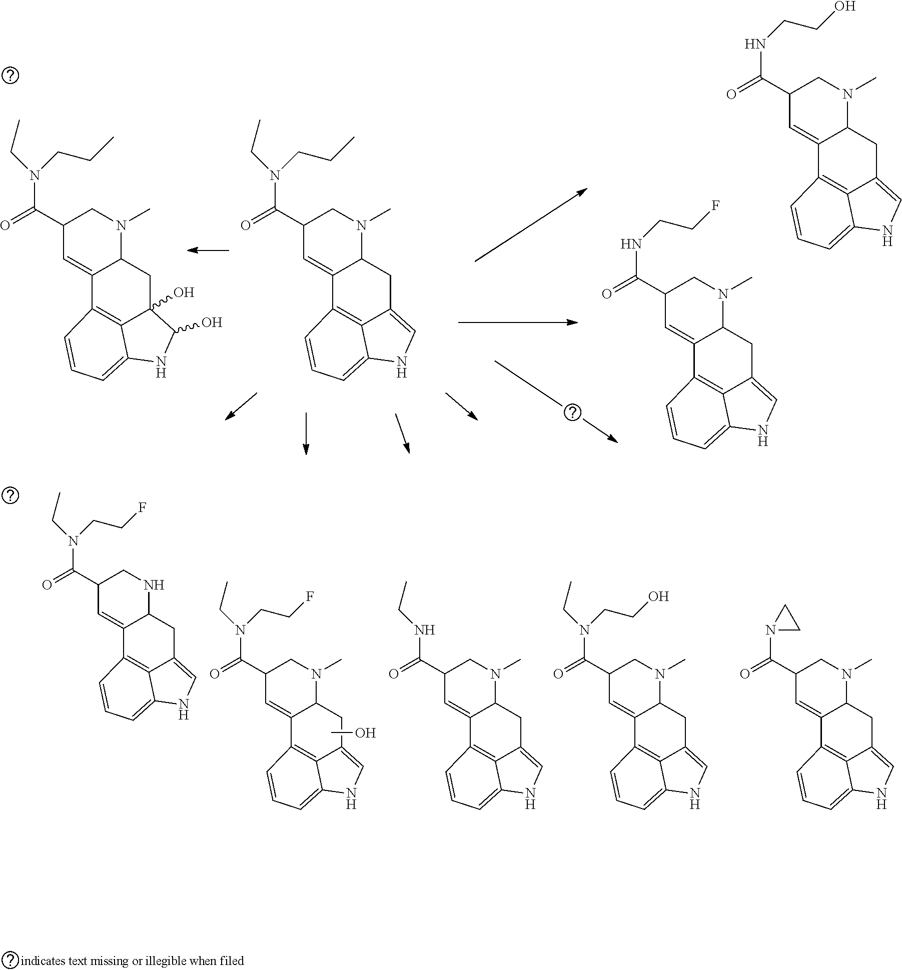

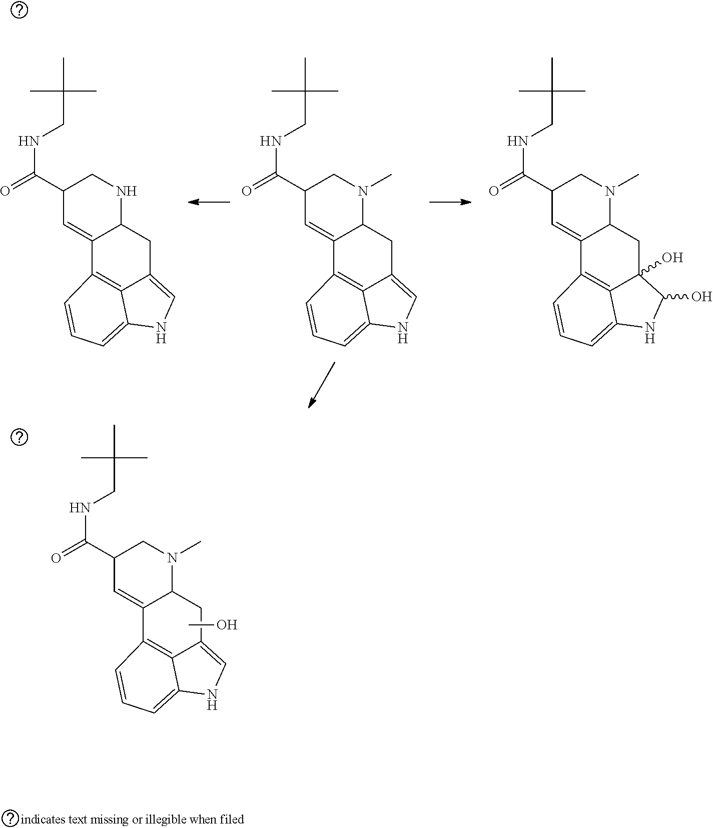

It has thus been shown that the novel LSD derivatives according to the invention, including the compounds L1, L2, L3 and L4, undergo a similar metabolization in human liver microsomes as LSD, and do not give rise to toxic metabolites. These findings further confirm the suitability of the LSD derivatives provided herein to be used in therapy.

1. A method for the production of lysergic acid diethylamide (LSD), comprising the steps of:
a. preparing a suspension of lysergic acid hydrate in ethyl acetate;
b. addition of diethylamine under protective gas atmosphere;
c. addition of propane-phosphonic acid anhydride solution (T3P) in ethyl acetate;
d. stirring of the mixture under protective gas atmosphere for at least 4 hours;
e. stopping the reaction by dilution with ethyl acetate;
f. extraction with water;
g. drying of the organic phase over a desiccant at 20-60° C. and under vacuum;
h. obtaining a crude product containing lysergic acid diethylamide (LSD).

2. A method for the production of a lysergic acid diethylamide (LSD) derivative of the following formula II 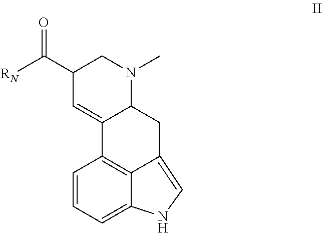
wherein:
R_{N} is selected from —NH—(C₁₋₅ alkyl), —N(C₁₋₅ alkyl)(C₁₋₅ alkyl), —NH—(C₁₋₅ haloalkyl), —N(C₁₋₅ alkyl)(C₁₋₅ haloalkyl), —N(C₁₋₅ haloalkyl)(C₁₋₅ haloalkyl), —NH—(C₁₋₅ alkylene)-O—(C₁₋₅ alkyl), —N(C₁₋₅ alkyl)[—(C₁₋₅ alkylene)-O—(C₁₋₅ alkyl)], —N[—(C₁₋₅ alkylene)-O—(C₁₋₅ alkyl)]-(C₁₋₅ alkylene)-O—(C₁₋₅ alkyl), —N(C₁₋₅ haloalkyl)[—(C₁₋₅ alkylene)-O—(C₁₋₅ alkyl)], —N(C₃₋₇ cycloalkyl)(C₃₋₇ cycloalkyl), an N-containing polycyclic heterocyclyl, 1,3-oxazolidin-3-yl, 3-methylpyrrolidin-1-yl, and an N-containing monocyclic heterocyclyl which is substituted with one or more halogens,
wherein any alkyl groups and/or any alkylene groups comprised in said —NH—(C₁₋₅ alkylene)-O—(C₁₋₅ alkyl), in said —N(C₁₋₅ alkyl)[—(C₁₋₅ alkylene)-O—(C₁₋₅ alkyl)] or in said —N[—(C₁₋₅ alkylene)-O—(C₁₋₅ alkyl)]-(C₁₋₅ alkylene)-O—(C₁₋₅ alkyl) are each optionally substituted with one or more halogens, wherein said N-containing polycyclic heterocyclyl or said N-containing monocyclic heterocyclyl comprises at least one nitrogen ring atom and is attached to the remainder of the compound via said nitrogen ring atom, wherein said N-containing polycyclic heterocyclyl is not indolin-1-yl, 1,2,3,4-tetrahydroquinolin-1-yl or 3-azabicyclo[3.2.2]nonan-3-yl, wherein said N-containing polycyclic heterocyclyl, said 1,3-oxazolidin-3-yl, said N-containing monocyclic heterocyclyl, and the cycloalkyl groups comprised in said —N(C₃₋₇ cycloalkyl)(C₃₋₇ cycloalkyl) are each optionally substituted with one or more groups R₄, and further wherein R_{N} is not —N(CH₂CH₃)—CH₂CH₃; and
each R₄ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—CO—O—(C₁₋₅ alkyl), —(C₀₋₃ alkylene)-N(C₁₋₅ alkyl)-CO—O—(C₁₋₅ alkyl), —(C₀₋₃ alkylene)-O—CO—NH—(C₁₋₅ alkyl), —(C₀₋₃ alkylene)-O—CO—N(C₁₋₅ alkyl)-(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO₂—(C₁₋₅ alkyl), and —(C₀₋₃ alkylene)-SO—(C₁₋₅ alkyl);
wherein the method comprises the steps of:
a. preparing a suspension of lysergic acid hydrate in ethyl acetate;
b. addition of an amine compound of the formula R_{N}—H, wherein R_{N} has the same meaning as in formula II, under protective gas atmosphere;
c. addition of propane-phosphonic acid anhydride solution (T3P) in ethyl acetate;
d. stirring of the mixture under protective gas atmosphere for at least 4 hours;
e. stopping the reaction by dilution with ethyl acetate;
f. extraction with water;
g. drying of the organic phase over a desiccant at 20-60° C. and under vacuum;
h. obtaining a crude product containing the LSD derivative of formula II.

3. The method according to claim 2, wherein the steps a. to f. are carried out at 25° C.

4. The method according to claim 3, wherein 10 equivalents of diethylamine are used.

5. The method according to claim 2, wherein the crude product is subsequently subjected to a method for isomer optimization, comprising the steps of:
a. dissolving the crude product in ethanol,
b. addition of sodium methoxide,
c. stirring for at least 2 hours,
d. dilution with water,
e. distillation of the solvent,
f. redilution of the residue with water,
g. extraction with ethyl acetate,
h. drying of the organic phase over a desiccant at 40-60° C. and under vacuum,
i. obtaining the isomer-optimized intermediate product.

6. The method according to claim 5, wherein the isomer-optimized intermediate product is subsequently subjected to a column purification process using a toluene/ethanol mixture.

7. (canceled)

8. A lysergic acid diethylamide (LSD) derivative produced by the method according to claim 2.

9. A compound which is a lysergic acid diethylamide (LSD) derivative according to the general formula I: 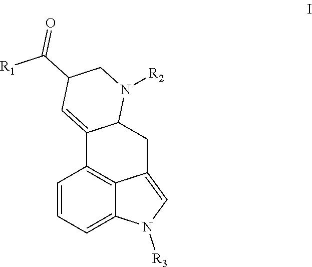
or a pharmaceutically acceptable salt thereof,
wherein:
R₁ is selected from —NH—(C₁₋₅ haloalkyl), —N(C₁₋₅ alkyl)(C₁₋₅ haloalkyl), —N(C₁₋₅ haloalkyl)(C₁₋₅ haloalkyl), —NH—CH₂—O—(C₁₋₅ alkyl), —NH—(CH₂)₃₋₅—O—(C₁₋₅ alkyl), —N(C₁₋₅ alkyl)[—(C₁₋₅ alkylene)-O—(C₁₋₅ alkyl)], —N[—(C₁₋₅ alkylene)-O—(C₁₋₅ alkyl)]-(C₁₋₅ alkylene)-O—(C₁₋₅ alkyl), —N(C₁₋₅ haloalkyl)[—(C₁₋₅ alkylene)-O—(C₁₋₅ alkyl)], —N(C₃₋₇ cycloalkyl)(C₃₋₇ cycloalkyl), an N-containing polycyclic heterocyclyl, 1,3-oxazolidin-3-yl, 3-methylpyrrolidin-1-yl, and an N-containing monocyclic heterocyclyl which is substituted with one or more halogens,
wherein said —NH—(C₁₋₅ haloalkyl) is not —NH—CH₂CH₂—Cl or —NH—CH(—CH₂CH₃)—CH₂—Cl, wherein any alkyl groups and/or any alkylene groups comprised in said —NH—CH₂—O—(C₁₋₅ alkyl), in said —NH—(CH₂)₃₋₅—O—(C₁₋₅ alkyl), in said —N(C₁₋₅ alkyl)[—(C₁₋₅ alkylene)-O—(C₁₋₅ alkyl)] or in said —N[—(C₁₋₅ alkylene)-O—(C₁₋₅ alkyl)]-(C₁₋₅ alkylene)-O—(C₁₋₅ alkyl) are each optionally substituted with one or more halogens, wherein said N-containing polycyclic heterocyclyl or said N-containing monocyclic heterocyclyl comprises at least one nitrogen ring atom and is attached to the remainder of the compound of formula I via said nitrogen ring atom, wherein said N-containing polycyclic heterocyclyl is not indolin-1-yl, 1,2,3,4-tetrahydroquinolin-1-yl or 3-azabicyclo[3.2.2]nonan-3-yl, and further wherein said N-containing polycyclic heterocyclyl, said 1,3-oxazolidin-3-yl, said N-containing monocyclic heterocyclyl, and the cycloalkyl groups comprised in said —N(C₃₋₇ cycloalkyl)(C₃₋₇ cycloalkyl) are each optionally substituted with one or more groups R₄, and
R₂ is selected from C₁₋₅ alkyl, C₂₋₅ alkenyl, C₂₋₅ alkynyl, and C₁₋₅ haloalkyl;
or alternatively R₁ is —NH—(C₁₋₅ alkyl) or —N(C₁₋₅ alkyl)(C₁₋₅ alkyl), and
R₂ is C₁₋₅ haloalkyl;
R₃ is selected from hydrogen, C₁₋₅ alkyl, —CO—(C₁₋₅ alkyl), —CO—(C₃₋₆ cycloalkyl), and an amino acid, wherein said amino acid is attached via a —CO— group formed from a carboxylic acid group of the amino acid, and further wherein said C₁₋₅ alkyl, the alkyl group comprised in said —CO—(C₁₋₅ alkyl), the cycloalkyl group comprised in said —CO—(C₃₋₆ cycloalkyl) and any alkyl group comprised in said amino acid are each optionally substituted with one or more halogens; and
each R₄ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-NO₂, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—CO—O—(C₁₋₅ alkyl), —(C₀₋₃ alkylene)-N(C₁₋₅ alkyl)-CO—O—(C₁₋₅ alkyl), —(C₀₋₃ alkylene)-O—CO—NH—(C₁₋₅ alkyl), —(C₀₋₃ alkylene)-O—CO—N(C₁₋₅ alkyl)-(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO₂—(C₁₋₅ alkyl), and —(C₀₋₃ alkylene)-SO—(C₁₋₅ alkyl).

10. The compound according to claim 9, wherein: R₁ is selected from —NH—(C₁₋₅ haloalkyl), —N(C₁₋₅ alkyl)(C₁₋₅ haloalkyl), —N(C₁₋₅ haloalkyl)(C₁₋₅ haloalkyl), —N(C₁₋₅ haloalkyl)[—(C₁₋₅ alkylene)-O—(C₁₋₅ alkyl)], —N(—CH₃)—CH₂CH₂—O—CH₃, —N(cyclopropyl)(cyclopropyl), an N-containing polycyclic heterocycloalkyl, 1,3-oxazolidin-3-yl, 3-methylpyrrolidin-1-yl, and an N-containing monocyclic heterocycloalkyl which is substituted with one or more halogens,
wherein said —NH—(C₁₋₅ haloalkyl) is not —NH—CH₂CH₂—Cl or —NH—CH(—CH₂CH₃)—CH₂—Cl, wherein said N-containing polycyclic heterocycloalkyl or said N-containing monocyclic heterocycloalkyl comprises at least one nitrogen ring atom and is attached to the remainder of the compound of formula I via said nitrogen ring atom, wherein said N-containing polycyclic heterocycloalkyl is not 3-azabicyclo[3.2.2]nonan-3-yl, and further wherein said N-containing polycyclic heterocycloalkyl, said 1,3-oxazolidin-3-yl, and said N-containing monocyclic heterocycloalkyl are each optionally substituted with one or more groups R₄; and
R₂ is selected from C₁₋₅ alkyl, C₂₋₅ alkenyl, C₂₋₅ alkynyl, and C₁₋₅ haloalkyl; preferably wherein R₂ is methyl or —CH₂CH₂F.

11. The compound according to claim 9, wherein R₁ is —NH—(C₁₋₅ alkyl) or —N(C₁₋₅ alkyl)(C₁₋₅ alkyl), and wherein R₂ is C₁₋₅ haloalkyl; preferably wherein R₂ is —CH₂CH₂F.

12. The compound according to claim 9, wherein R₃ is hydrogen, —CO—(C₁₋₅ alkyl), or —CO—(C₃₋₆ cycloalkyl).

13. The compound according to claim 9, wherein:
R₁ is selected from 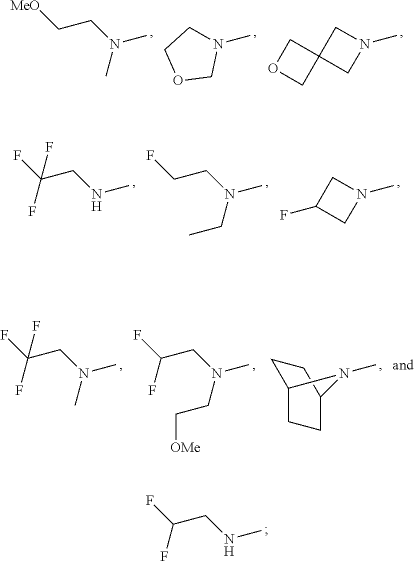
R₂ is selected from —CH₃ and —CH₂CH₂F; and
R₃ is selected from —H, —COCH₃ and —COCH₂CH₃;
or alternatively R₁ is 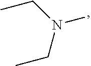
R₂ is —CH₂CH₂F, and R₃ is selected from —H, —COCH₃ and —COCH₂CH₃.

14. The compound according to claim 9, wherein said compound has the following absolute configuration:

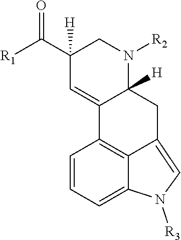

15. The compound according to claim 9, wherein said compound is selected from any one of the following compounds: 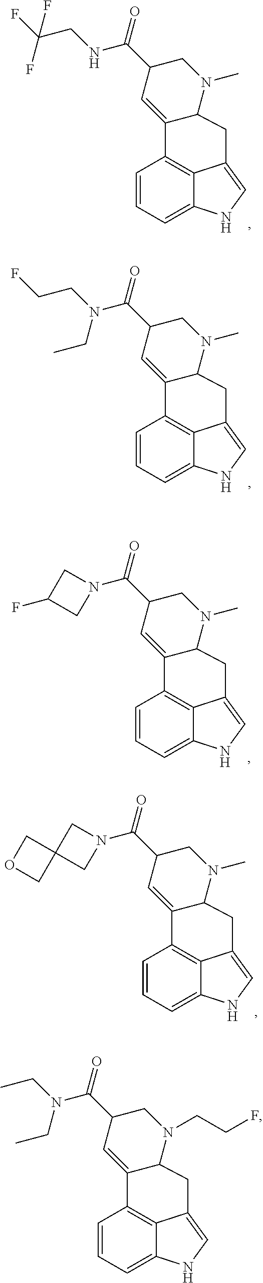 [SMILES: CN1CC(C(=O)NCC(F)(F)F)C=C2c3cccc4[nH]cc(c34)CC21 ; CCN(CCF)C(=O)C1C=C2c3cccc4[nH]cc(c34)CC2N(C)C1 ; CN1CC(C(=O)N2CC(F)C2)C=C2c3cccc4[nH]cc(c34)CC21 ; CN1CC(C(=O)N2CC3(COC3)C2)C=C2c3cccc4[nH]cc(c34)CC21 ; CCN(CC)C(=O)C1C=C2c3cccc4[nH]cc(c34)CC2N(CCF)C1] 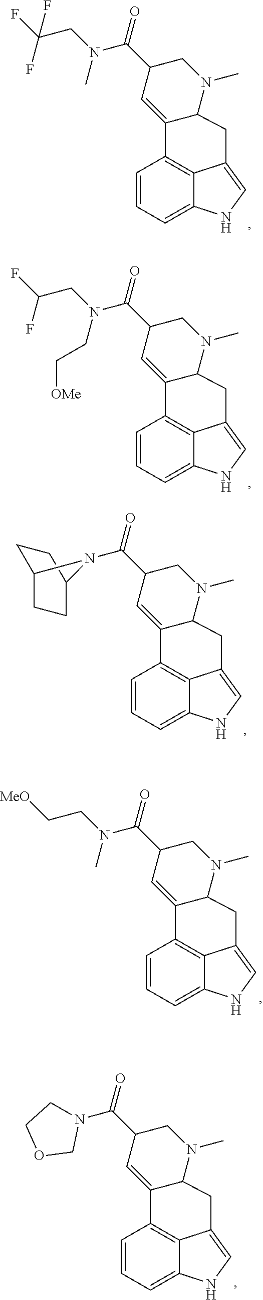 [SMILES: CN(CC(F)(F)F)C(=O)C1C=C2c3cccc4[nH]cc(c34)CC2N(C)C1 ; COCCN(CC(F)F)C(=O)C1C=C2c3cccc4[nH]cc(c34)CC2N(C)C1 ; CN1CC(C(=O)N2C3CCC2CC3)C=C2c3cccc4[nH]cc(c34)CC21 ; COCCN(C)C(=O)C1C=C2c3cccc4[nH]cc(c34)CC2N(C)C1 ; CN1CC(C(=O)N2CCOC2)C=C2c3cccc4[nH]cc(c34)CC21] 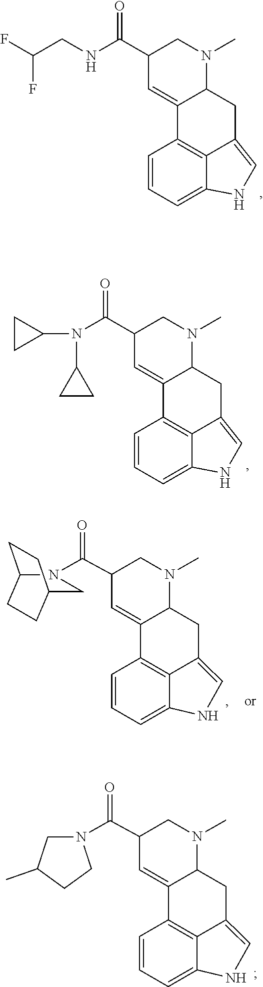 [SMILES: CN1CC(C(=O)NCC(F)F)C=C2c3cccc4[nH]cc(c34)CC21 ; CN1CC(C(=O)N(C2CC2)C2CC2)C=C2c3cccc4[nH]cc(c34)CC21 ; CN1CC(C(=O)N2CC3CCC2CC3)C=C2c3cccc4[nH]cc(c34)CC21 ; CC1CCN(C(=O)C2C=C3c4cccc5[nH]cc(c45)CC3N(C)C2)C1]
or a pharmaceutically acceptable salt thereof.

16. A pharmaceutical composition comprising the lysergic acid diethylamide (LSD) derivative according to claim 8, and optionally one or more pharmaceutically acceptable excipients.

17. (canceled)

18. (canceled)

19. (canceled)

20. (canceled)

21. (canceled)

22. (canceled)

23. (canceled)

24. (canceled)

25. (canceled)

26. A method of treating a serotonin 5-HT_{2A} receptor associated disease/disorder in a subject in need thereof, the method comprising administering a therapeutically effective amount of the lysergic acid diethylamide (LSD) derivative according to claim 8 to said subject.

27. The method according to claim 26, wherein said disease/disorder is an anxiety disorder, attention deficit hyperactivity disorder (ADHD), depression, cluster headache, a condition associated with cancer, diminished drive, burn-out, bore-out, migraine, Parkinson's disease, dementia, pulmonary hypertension, schizophrenia, an eating disorder, nausea, or vomiting.

28. (canceled)

29. The method according to claim 27, wherein said disease/disorder is an anxiety disorder, attention deficit hyperactivity disorder (ADHD), depression, cluster headache, a condition associated with cancer, diminished drive, burn-out, bore-out, migraine, Parkinson's disease, dementia, pulmonary hypertension, schizophrenia, an eating disorder, nausea, or vomiting.

30. (canceled)
